(19) United States
(12) Patent Application Publication
(10) Pub. No.: US 2010/0242188 A1
(43) Pub. Date: Sep. 30, 2010

(54) COMPOSITION FOR COLOURING KERATINOUS MATERIAL USING A SUPRA-MOLECULAR COLOURING SYSTEM

(76) Inventors: Nicolas Daubresse, La Celles ST Cloud (FR); Timo Luukas, Massy (FR); Sandrine Chodorowski-Kimmes, Senlis (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/447,134

(22) PCT Filed: Oct. 25, 2007
(86) PCT No.: PCT/FR2007/001767

§ 371 Date: Jun. 1, 2010

Related U.S. Application Data
(60) Provisional application No. 60/861,038, filed on Nov. 27, 2006.

(30) Foreign Application Priority Data
Oct. 25, 2006 (FR) 0654515

(51) Int. Cl. A61K 8/49; A61Q 5/10

(52) U.S. Cl. 8/425

(57) ABSTRACT

The present invention relates to a composition for colouring keratinous materials. The composition contains in an appropriate medium at least one supra-molecular colouring system of the type (COLORANT)ₙ(SAU)ₘ(I), in which (COLORANT) is any chromophore having a maximal absorption of between 300 nm and 1200 nm covalently bonded to (SAU), and (SAU) represents an acceptor/donor radical capable of forming at least three hydrogen bonds. The invention provides a resistant and varied colouration of keratinous materials.

The subject of the present invention is a cosmetic composition for colouring keratinous material, containing, in an appropriate medium, at least one supra-molecular colouring system, in particular a cosmetic composition for colouring human keratin fibres such as the hair, or a composition for making up keratinous material such as the lips, the eyelashes, the skin and the nails.

For a long time, many individuals have been seeking to modify the colour of their hair, and in particular to mask their white hair.

To do this, it is known practice to dye keratin fibres permanently by means of oxidation dyeing. This colouring technique consists in applying to the keratin fibres a composition containing dye precursors such as oxidation bases and couplers. These precursors, under the action of an oxidizing agent, will form one or more coloured species in the hair.

The variety of molecules available for use in terms of oxidation bases and couplers makes it possible to obtain a rich palette of colours. The colorations which result therefrom are permanent, powerful, and resistant to external agents, in particular to light, bad weather, washing, perspiration and rubbing.

However, the colouring is carried out using oxidizing products, such as aqueous hydrogen peroxide, in a basic medium. These oxidizing agents attack the keratin of the hair, the cosmetic and mechanical properties of which may greatly degrade in the case of repeated colorations, which may lead to difficult hair styling or hair shaping.

Moreover, it is known practice to dye keratin fibres, and in particular human hair, with colouring compositions containing direct dyes. The conventional dyes that are used are, in particular, dyes of the nitrobenzene, anthraquinone, nitropyridine, azo, xanthene, acridine, azine or triarylmethane type, or natural dyes. These dyes may be nonionic, anionic, cationic or amphoteric. These dyes are coloured and colouring molecules having an affinity for keratin fibres.

The colorations which result therefrom are particularly chromatic colorations which are, however, temporary or semi-permanent since the nature of the interactions which bind the direct dyes to the keratin fibre, and the desorption thereof from the surface and/or from the core of the fibre, are responsible for weak resistance to washing or to perspiration.

There exists a need to develop new colouring compositions for obtaining varied chromatic or natural shades, which have improved dyeing properties, such as good resistance, in particular to external agents such as light, shampoo or sweat. In particular, there exists a need to develop colouring compositions for obtaining colorations having a resistance close to oxidation dyeing, without the drawbacks associated with the presence of an oxidizing agent.

Furthermore, individuals have always been seeking to modify the colour of keratinous material, such as the skin, the lips, the eyelashes and the nails, for aesthetic reasons. In the make-up field, there also exists a need to develop new compositions which make it possible to obtain a make-up result that is more resistant to external agents, while at the same time respecting the physiology of the keratinous material.

These objectives are achieved by means of the present invention, the subject of which is a cosmetic composition for colouring keratinous material, containing, in an appropriate medium, a supra-molecular colouring system, the colouring system comprising either
A) at least one dye of formula (I) below:

(DYE)ₙ(SAU)ₘ (I)
in which:
(DYE) represents any chromophore having a maximum absorption between 300 nm and 1200 nm, covalently bonded to (SAU),
(SAU) represents an acceptable/donor radical capable of forming at least three hydrogen bonds, and capable of self-assembly under appropriate conditions,
or
(B) at least one dye according to general formula (I) defined above and at least one other analogous dye (I′) of formula

(DYE′)_{n′}(SAU′)_{m′}
in which DYE′ and SAU′ have the same definitions as DYE and SAU, and the (SAU) radicals being capable of assembling with the (SAU′) radicals under appropriate conditions,
or
(C) at least one dye according to general formula (I) and/or (I′) defined above, and at least one compound according to formula (II):

(SAU″)ₚ(X)_{q} (II)
in which SAU″ has the same definition as SAU, the (SAU) or (SAU′) radicals being capable of assembling with the (SAU″) radicals under appropriate conditions,
   n, n′, m, m′ and p are integers greater than or equal to 1; and q is equal to 0 or 1,
   and also the addition salts and solvates thereof.

When n or n′ is greater than 1, the (DYE) or (DYE′) radicals may be identical or different. When m or m′ or p is greater than 1, the (SAU), (SAU′) or (SAU″) radicals may be identical or different.

Through the use of the supra-molecular colouring systems of the type of those described above, a composition which makes it possible to obtain a coloration of keratinous material having good dyeing properties, while at the same time respecting this keratinous material, such as the hair, the eyelashes, the lips, the nails or the skin, is obtained. Moreover, such a composition makes it possible to obtain a coloration which is resistant but which can also be readily eliminated by breaking the noncovalent interactions linking the various dyes of formulae (I) and/or (I′).

In particular, the composition of the invention makes it possible to obtain better properties such as stability to light, to water, to perspiration and to washing, and also easy elimination of the colour due to the reversibility of the interactions existing in such a molecular system.

These noncovalent interactions are often dominated by a hydrogen bond and, if aromatic components are present, by π interactions between the aromatic nuclei. Weak forces such as Van der Waals interactions can also act.

The dyes of formula (I) and/or (I′) and the compounds of formula (II) that are of use in the composition of the invention are, for example, described in patent applications EP 1 310 533 and EP 1 486 539.

According to the present invention, the self-assembly process can take place between the dyes (I) and/or (I′), but also between the dyes of formula (I) and/or (I′) and compounds of formula (II) which do not have a dye fragment.

By way of chromophores that are of use in the present invention, mention may be made of the radicals derived from acridine, acridone, anthranthrone, anthrapyrimidine, anthraquinone, azine, azo, azomethine, benzanthrone, benzimidazole, benzimidazolone, benzindole, benzoxazole, benzopyran, benzothiazole, benzoquinone, bisazine, bisisoindoline, carboxanilide, coumarin, cyanin (azacarbocyanin, diazacarbocyanin, diazahemicyanin, hemicyanin, tetraazacarbocyanin), diazine, diketopyrrolopyrrole, dioxazine, diphenylamine, diphenylmethane, dithiazine, flavonoid, such as flavanthrone and flavone, fluorindine, formazan, hydrazone, in particular arylhydrazone, hydroxyketone, indamine, indanthrone, indigoid and pseudoindigoid, indophenol, indoaniline, isoindoline, isoindolinone, isoviolanthrone, lactone, methine, naphthalimide, naphthanilide, naphtholactam, naphthoquinone, nitro, in particular nitro(hetero)aromatic, oxadiazole, oxazine, perilone, perinone, perylene, phenazine, phenothiazine, phthalocyanin, polyene/carotenoid, porphyrin, pyranthrone, pyrazolanthrone, pyrazolone, pyrimidinoanthrone, pyronine, quinacridone, quinoline, quinophthalone, squarane, stilbene, thiazine, thioindigo, thiopyronine, triarylmethane and xanthene dyes.

Among the nitro chromophores that can be used according to the invention, mention may be made, in a nonlimiting manner, of the radicals derived from the following dyes:
1,4-diamino-2-nitrobenzene
1-amino-2 nitro-4-β-hydroxyethylaminobenzene
1-amino-2 nitro-4-bis(β-hydroxyethyl)aminobenzene
1,4-bis(β-hydroxyethylamino)-2-nitrobenzene
1-β-hydroxyethylamino-2-nitro-4-bis-(β-hydroxyethylamino)benzene
1-β-hydroxyethylamino-2-nitro-4-aminobenzene
1-hydroxyethylamino-2-nitro-4-(ethyl) (β-hydroxyethyl)aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis-(β-hydroxyethylamino)-4-nitrobenzene
1-amino-2-tris-(hydroxymethyl)methylamino-5-nitrobenzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-β-hydroxyethylamino-5-nitrobenzene
1-methoxy-2-hydroxyethylamino-5-nitrobenzene
1-β-hydroxyethyloxy-3-methylamino-4-nitrobenzene
1-β,γ-dihydroxypropyloxy-3-methylamino-4-nitrobenzene
1-β-hydroxyethylamino-4-β,γ-dihydroxypropyloxy-2-nitrobenzene
1-β,γ-dihydroxypropylamino-1-trifluoromethyl-2-nitrobenzene
1-β-hydroxyethylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-ethylamino-4-nitrobenzene
1-hydroxy-2-chloro-6-amino-4-nitrobenzene
1-hydroxy-6-bis-(β-hydroxyethyl)amino-3-nitrobenzene
1-β-hydroxyethylamino-2-nitrobenzene
1-hydroxy-4-β-hydroxyethylamino-3-nitrobenzene.

Mention may also be made, among the azo chromophores, of the following compounds described in the Colour Index International 3rd edition:

Disperse Red 17

Acid Yellow 9

Acid Black 1

Acid Yellow 36

Acid Orange 7

Acid Red 33

Acid Red 35

Acid Yellow 23

Acid Orange 24

Disperse Black 9.

Mention may also be made of 1-(4′-aminodiphenylazo)-2-methyl-4-bis(β-hydroxyethyl)amino-benzene and 4-hydroxy-3-(2-methoxyphenylazo)-1-naphthalenesulphonic acid.

Among the quinone chromophores, mention may be made of the radicals derived from the following dyes:

Disperse Red 15

Solvent Violet 13

Acid Violet 43

Disperse Violet 1

Disperse Violet 4

Disperse Blue 1

Disperse Violet 8

Disperse Blue 3

Disperse Red 11

Acid Blue 62

Disperse Blue 7

Disperse Violet 15

and also the following compounds:
1-aminopropylamino-4-methylaminoanthraquinone
1-aminopropylaminoanthraquinone
5-β-hydroxyethyl-1,4-diaminoanthraquinone
2-aminoethylaminoanthraquinone
1,4-bis(β,γ-dihydroxypropylamino)anthraquinone.

Among the indoamine chromophores that can be used according to the invention, mention may be made of the radicals derived from the following dyes:
2-β-hydroxyethylamino-5-[bis(β-4′-hydroxyethyl)amino]anilino-1,4-benzoquinone
2-β-hydroxyethylamino-5-(2′-methoxy-4′-amino)anilino-1,4-benzoquinone
3-N(2′-chloro-4′-hydroxy)phenylacetylamino-6-methoxy-1,4-benzoquinoneimine
3-N(3′-chloro-4′-methylamino)phenylureido-6-methyl-1,4-benzoquinoneimine
3-[4′-N-(ethyl,carbamylmethyl)amino]phenylureido-6-methyl-1,4-benzoquinoneimine.

Mention may also be made of those described in the encyclopaedia “The chemistry of synthetic dye” by K. Venkataraman, 1952, Academic Press Vol. 1 to 7, in the encyclopaedia “Kirk Othmer” “Chemical technology”, Chapter “Dyes and dye intermediate”, 1993, Wiley & Sons, and in various chapters of the encyclopaedia “Ullmann's Encyclopaedia of Industry Chemistry” 7th Edition, Wiley & Sons.

The preferred chromophores are those which absorb light at between 380 nm and 850 nm.

The hydrogen bonds formed by “SAU” or “SAU′” or “SAU″” represent a special type of electrostatic interaction and can be described as an attractive interaction between a proton donor and a proton acceptor.

Thus, the “SAU” or “SAU′” or “SAU″” radical is an acceptable/donor radical which can form at least three hydrogen bonds, preferably 3 or 4 hydrogen bonds.

Preferably, the (SAU) or (SAU′) or (SAU″) radical can form 4 hydrogen bonds with an identical partner group, among which are 2 donor bonds, for example NH, and 2 acceptor bonds, for example CO and —C═N—.

Many examples of “SAU” or “SAU′” radicals can be found, without this being limiting, in the publications: Chem. Soc. Rev., 2001, 30, pages 83 to 93; Tetrahedron, 57 (2001), pages 1139 to 1159; J. Am. Chem. Soc., 2001, 123, pages 409 to 416; Adv. Mater., 2000, 12, No. 12, pages 874 to 878; Chem. Eur. J., 2001, 7, No. 10, pages 2059 to 2065; J. Am. Chem. Soc., 2000, 122, pages 5006 to 5007; Chem. Eur. J., 2000, 6, No. 21 pages 3871 to 3886; Tetrahedron, 56 (2000), pages 8419 to 8427; WO 98/14504; Monographs in Supramolecular Chemistry, No. 7, Self-Assembly in Supramolecular Systems, L. F. Lindoy, I. M. Atkinson, in particular the examples mentioned in Chapter 3; New Polymers based on the Quadruple Hydrogen Bonding Motif, Brigitte J. B. Folmer, thèse de Ph. D. [Ph. D thesis], June 2000, T U Eindhoven; J. Org. Chem., 2001, 66 pages 6513 to 6522; Tetrahedron Letters, 42 (2001), pages 7357 to 7359; Chemistry Letters, 2001, 7, page 694.

Preferably, the (SAU) or (SAU′) or (SAU″) radical comprises rings containing 5 or 6 atoms, it being possible for these rings to be (hetero)aromatic or unsaturated heterocycles. According to one particular embodiment, these rings are constituted of C and/or N atoms, and with conjugated double bonds for stabilizing and directing the H interactions.

According to another embodiment, the (SAU) or (SAU′) or (SAU″) radical comprises rings containing 6 atoms comprising C and/or N atoms and with conjugated double bonds for stabilizing and directing the H interactions.

The (SAU) or (SAU′) or (SAU″) radical is preferably chosen from the radicals derived from the compounds:
aminopyrimidones, ureidopyrimidones,
trimellitic acid,
acylaminopyridines, ureidopyridines, carbamoyl-pyridines,
acylaminotriazines, ureidotriazines and in particular ureidoaminotriazines, acylaminoamino-triazines,
acylaminotriazoles,
urazoylbenzoic acid,
phthalhydrazides,
2-aminopyrimidine-4,6(1H,5H)-diones, and/or 4,6-diaminopyrimidin-2(1H)-ones substituted respectively on their 5 position with a linear, branched or cyclic, C₁-C₂₀ saturated or unsaturated, optionally aromatic, carbon-based, in particular alkylene, radical which may contain one or more heteroatoms; and the derivatives of these compounds and also the tautomeric forms.

Among these compounds, preference is given, for the (SAU) or (SAU′) or (SAU″) radical, to:
ureidopyrimidones;
ureidopyridines, carbamoylpyridines;
acylaminoamino-s-triazines;
ureidotriazines;
phthalhydrazides;
2-aminopyrimidine-4,6(1H,5H)-diones, and/or 4,6-diaminopyrimidine-2(1H)-ones;

and derivatives thereof.

According to one particular embodiment, when the (SAU) or (SAU′) or (SAU″) radicals are different and present in the same colouring composition, they are in particular chosen from the following combinations
adenine/guanine;
cytidine/thymine;
pyridine/phenol;
triamino-s-triazine/uracil or succinimide or glutarimide or cyanuric acid or thymine or maleimide or (di)aminopyrimidine or barbituric acid;
acylaminoamino-s-triazine/uracil or succinimide or glutarimide or cyanuric acid or thymine or maleimide or (di)aminopyrimidine or barbituric acid;
   and the derivatives thereof and the tautomeric forms thereof.

The (SAU) or (SAU′) or (SAU″), “DYE” or “DYE′”, and X radicals may comprise one or more linker arms L as a function of the values of n, n′, m, m′, p, q, such as

linear or branched, optionally substituted alkylene radicals;

optionally substituted cycloalkylene radicals;

optionally substituted arylene radicals;

saturated or unsaturated heterocyclic radicals;

amino (—NH— or —NR—) or alternatively —O—, —SO—, —SO₂— or —C(O)— radicals;

and also combinations thereof of the same category and/or of different categories, resulting in particular in cycloalkylenealkylene, biscycloalkylene, biscyclo-alkylenealkylene, arylenealkylene, bisphenylene-alkylene, oxyalkylene and aminoalkylene radicals.

These radicals may optionally be substituted, in particular with one or more C₁-C₁₂ alkyl groups, optionally comprising heteroatoms chosen from O, N, S, F and P, and combinations thereof.

Among the optionally substituted alkylene radicals, mention may, for example, be made of C₁-C₃₀ alkylene radicals, for example the methylene radical —CH₂— and poly(methylene) radicals of formula —(CH₂)ₙ— (n≧2), such as ethylene, butylene, in particular 1,4-butylene, and 1,6-hexylene radicals, and C₃-C₁₀ branched alkylene radicals such as 1,4-(4-methylpentylene), 1,6-(2,2,4-trimethylhexylene), 1,5-(5-methylhexylene), 1,6-(6-methylheptylene), 1,5-(2,2,5-trimethylhexylene), 1,7-(3,7-dimethyl-octylene), 2,2-(dimethylpropylene) and 1,6-(2,4,4-trimethylhexylene) radicals.

Among the optionally substituted cycloalkylene radicals, mention may, for example, be made of cyclopentylene and cyclohexylene radicals, which are optionally substituted, in particular with alkyl groups.

Among the cycloalkylenealkylenes, mention may, for example, be made of the isophorone radical of formula:

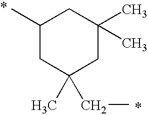

Among the optionally substituted biscycloalkylenealkylene radicals, mention may, for example, be made of the radicals of formula:

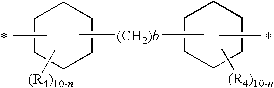

wherein b is an integer from 0 to 3, n an integer from 0 to 4; and R4, which may be identical or different, represents H or a C₁-C₁₂ alkyl radical, in particular a methyl radical; mention may in particular be made of 4,4′-methylenebiscyclohexylene.

Among the optionally substituted arylene radicals, mention may, for example, be made of the phenylene radical, tolylene radicals, in particular 2,4- and 2,6-tolylene radicals, and naphthylene radicals, in particular 2,4-naphthylene or 2,6-naphthylene.

Among the optionally substituted arylenealkylene radicals, mention may, for example, be made of phenylenealkylene radicals such as the benzylene radical:

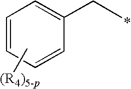

in which p is an integer from 0 to 5.

Among the optionally substituted bisphenylene-alkylene radicals, mention may, for example, be made of the radicals of formula (i):

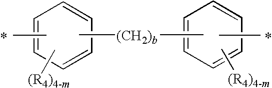

wherein b is an integer from 0 to 3, and m an integer from 0 to 4; such as the bisphenylene radical and the 4,4′-methylenebisphenylene radical,
and the radicals of formula (ii):

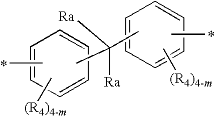

in which m is an integer from 0 to 4, and the Rₐ radicals, which may be identical or different, represent H or a C₁-C₄ alkyl radical, preferably a methyl radical.

Among the optionally substituted oxyalkylene radicals, mention may, for example, be made of the alkylene oxide radicals of formula —O—(R′O)_{y}— wherein R′, which may be identical or different, represents a linear or branched C₂-C₄ alkylene radical, in particular ethylene or propylene; and y is an integer from 1 to 500, preferably from 1 to 200, and more preferably from 5 to 100.

By way of example of linker arms, mention may be made of methylene, ethylene, linear or branched propylene, linear or branched butylene, linear or branched pentylene and linear or branched hexylene radicals, optionally substituted and/or interrupted as indicated above.

By way of examples of an aromatic or nonaromatic, saturated or unsaturated ring or heterocycle interrupting the alkyl radical of the linker arm, mention may be made of phenylene or naphthylene, phenanthrylene, triazinyl, pyrimidinyl, pyridinyl, pyridazinyl, quinoxalinyl or cyclohexyl radicals.

By way of examples of linker arms, mention may be made of:

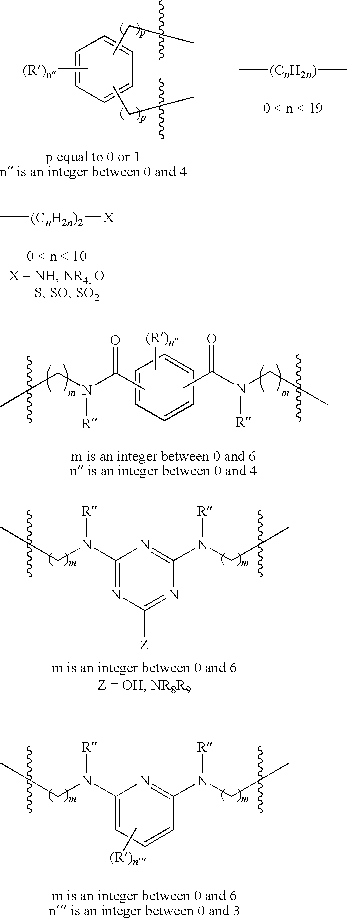

in which formulae:

R′ represents:
an optionally substituted C₁-C₁₆ alkyl radical, optionally interrupted with one or more heteroatoms or with one or more groups comprising at least one heteroatom, preferably chosen from oxygen, nitrogen and sulphur, such as —CO, —SO₂ or combinations thereof;
a hydroxyl group;
a C₁-C₄ alkoxy group;
a C₂-C₄ (poly)hydroxyalkoxy group;
an alkoxycarbonyl group (RO—CO—) in which R represents a C₁-C₄ alkyl radical;
an alkylcarbonyloxy radical (RCO—O—) in which R represents a C₁-C₄ alkyl radical;
an alkylcarbonyl radical (R—CO—) in which R represents a C₁-C₄ alkyl radical;
an amino group;
an amino group substituted with one or two C₁-C₄ alkyl radicals, which may be identical or different, optionally bearing at least one hydroxyl group; it being possible for the two alkyl radicals to optionally form, with the nitrogen atom to which they are attached, a saturated or unsaturated, aromatic or nonaromatic, optionally substituted heterocycle containing 1 to 3 heteroatoms, preferably 1 to 2 heteroatoms, chosen from N, O and S, preferably N, comprising from 5 to 7 ring members;
an alkylcarbonylamino group (RCO—NR′—) in which the R radical represents a C₁-C₄ alkyl radical and the R′ radical represents a hydrogen atom or a C₁-C₄ alkyl radical;
an aminocarbonyl group ((R)₂N—CO—) in which the R radicals, independently of one another, represent a hydrogen atom or a C₁-C₄ alkyl radical;
a ureido group (N(R)₂—CO—NR′—) in which the R and R′ radicals, independently of one another, represent a hydrogen atom or a C₁-C₄ alkyl radical;
an aminosulphonyl group ((R)₂N—SO₂—) in which the R radicals, independently of one another, represent a hydrogen atom or a C₁-C₄ alkyl radical;
an alkylsulphonylamino group (RSO₂—NR′—) in which the R and R′ radicals, independently of one another, represent a hydrogen atom or a C₁-C₄ alkyl radical;
an alkylthio group (RS—) in which the R radical represents a C₁-C₄ alkyl radical;
an alkylsulphinyl group (R—SO—) in which R represents a C₁-C₄ alkyl radical;
an alkylsulphonyl group (R—SO₂—) in which R represents a C₁-C₄ alkyl radical;
a nitro group;
a cyano group;
a halogen atom, preferably chlorine or fluorine;
R″, which are identical, represent a hydrogen or a C₁-C₄ alkyl radical.

R₈ and R₉ represent, independently of one another, a hydrogen atom, or a C₁-C₈ alkyl radical optionally substituted with one or more radicals, which may be identical or different, chosen from hydroxyl, C₁-C₂ alkoxy, C₂-C₄ (poly)hydroxyalkoxy, amino, C₁-C₂ (di)alkylamino or aryl, which is optionally substituted.

Preferably, “DYE”, “DYE′”, “SAU”, “SAU′” and “SAU″” comprise one to three linker arms.

The acid addition salts of supra-molecular dyes of the invention of formula (I) or (I′) may, by way of example, be the addition salts with an organic or inorganic acid such as hydrochloric acid, hydrobromic acid, sulphuric acid or (alkyl- or phenyl-)sulphonic acids, such as p-toluenesulphonicor acid methylsulphonic acid, or the salts of carboxylic acids, for instance acetates, lactates, benzoates, salicylates or citrates.

The solvates of the supra-molecular colouring systems of the invention are, for example, the hydrates of such systems or the combination of a compound of formula (I), (I′) or (II), (II′) with a C₁-C₄ linear or branched alcohol, such as methanol, ethanol, isopropanol or n-propanol.

Representative but nonlimiting examples of the various classes of supra-molecular colouring systems that are useful in the composition of the invention are presented in formulae 1 to 10 below. In certain formulae, the supra-molecular colouring system is represented, in others, the dyes are represented in their singular molecular form. Examples are given in Table 1.

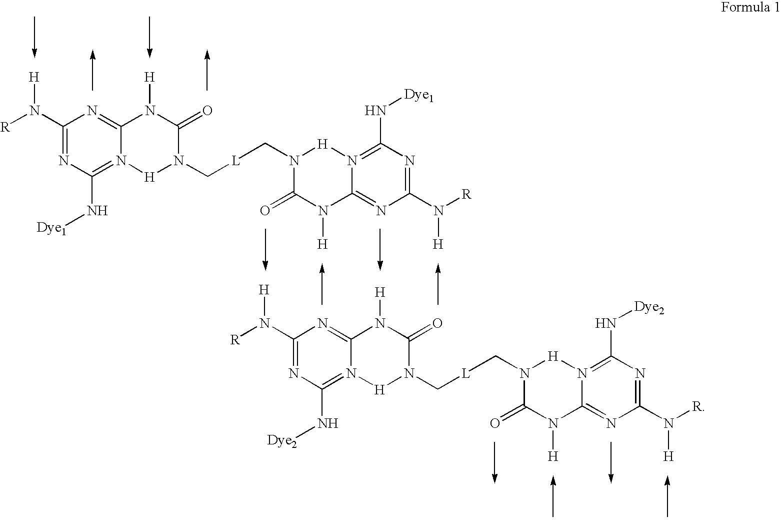

in which
the Dye₁ and Dye₂ groups are as defined for “Dye”, and R represents a hydrogen atom, a substituted or unsubstituted alkyl group, a substituted or unsubstituted aryl group, a substituted or unsubstituted aralkyl group, a substituted or unsubstituted acyl group, a substituted or unsubstituted sulphonyl group, a substituted or unsubstituted phosphoryl group or a heterocyclic group, and L represents a linker arm as defined above.

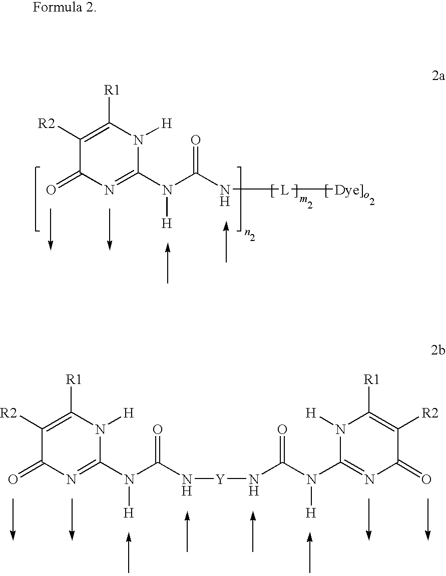

in which “L” represents a linker arm;
“Dye” is as defined above;
n₂ and o₂ are identical or different and greater than or equal to 1; m₂ can be greater than or equal to 0;
R1 and R2 are identical or different and represent a hydrogen atom, a halogen atom, a substituted or unsubstituted alkoxy group, a substituted or unsubstituted thioalkoxy group, a substituted or unsubstituted sulphoxy group, a substituted or unsubstituted sulphone group, a substituted or unsubstituted amino group, a nitrile group, a substituted or unsubstituted, saturated or unsaturated alkyl group, a substituted or unsubstituted acyl group, a substituted or unsubstituted sulphonyl group, a substituted or unsubstituted aryl group, a substituted or unsubstituted aralkyl group, a heterocyclic group or a “DYE” group, or R1 and R2 represent the atoms necessary for forming a system with a nucleus;
Y represents a divalent DYE group;

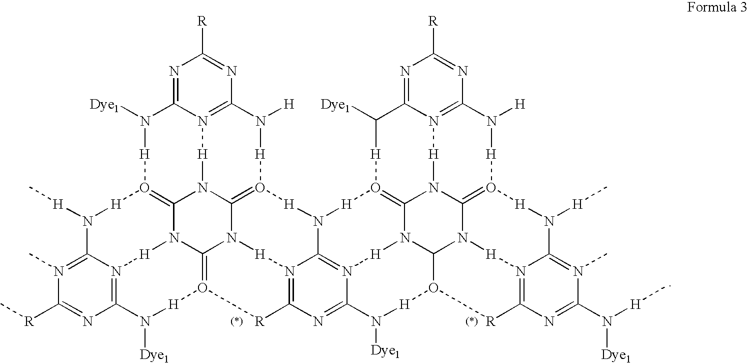

in which R represents a hydrogen atom, a substituted or unsubstituted alkyl group, a substituted or unsubstituted aryl group, a substituted or unsubstituted aralkyl group, a heterocyclic group, a dye, OR1 or NR2R3;
R1 represents a hydrogen atom, a substituted or unsubstituted alkyl group, a substituted or unsubstituted acyl group, a substituted or unsubstituted sulphonyl group, a substituted or unsubstituted aryl group, a substituted or unsubstituted aralkyl group, a heterocyclic group, or a Dye₁ or Dye₂ group;
R2 and R3 are identical or different and represent a hydrogen atom, a substituted or unsubstituted alkyl group, a substituted or unsubstituted aryl group, a substituted or unsubstituted aralkyl group, a heterocyclic group, a substituted or unsubstituted acyl group, a substituted or unsubstituted sulphonyl group, or a Dye₁ or Dye₂ group, or R2 and R3 represent the atoms necessary for forming, with the nitrogen atom, a ring;
the Dye₁ and Dye₂ groups are as defined for DYE.

When R2 and/or R3 represent(s) a hydrogen atom, then an additional hydrogen bond is formed in formula 3.

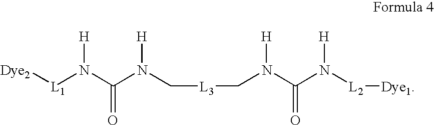

The Dye₁ and Dye₂ groups are as defined for DYE. L₁, L₂ and L₃ are identical or different and represent a linker arm as defined above.

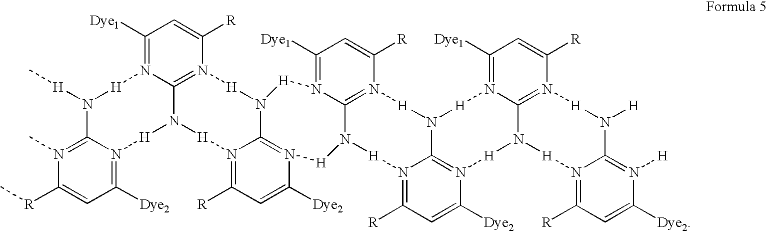

in which

R represents a hydrogen atom, a substituted or unsubstituted alkyl group, a substituted or unsubstituted aryl group, a substituted or unsubstituted aralkyl group, a heterocyclic group, a Dye₁ or Dye₂ group, OR1 or NR2R3;

R1 represents a hydrogen atom, a substituted or unsubstituted alkyl group, a substituted or unsubstituted aryl group, a substituted or unsubstituted aralkyl group or a heterocyclic group;

R2 and R3 are identical or different and represent a hydrogen atom, a substituted or unsubstituted alkyl group, a substituted or unsubstituted aryl group, a substituted or unsubstituted aralkyl group or a heterocyclic group, or R2 and R3 represent the atoms necessary for forming, with the nitrogen atom, a ring;

The Dye₁ and Dye₂ groups are as defined for DYE.

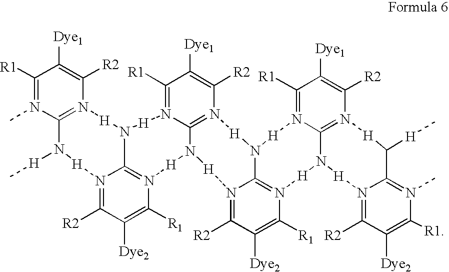

R1 and R2 are identical or different and represent a hydrogen atom, a substituted or unsubstituted alkyl group, a substituted or unsubstituted aryl group, a substituted or unsubstituted aralkyl group, a heterocyclic group, OR3 or NR4R5;

R3 represents a hydrogen atom, a substituted or unsubstituted alkyl group, a substituted or unsubstituted aryl group, a substituted or unsubstituted aralkyl group or a heterocyclic group;

R4 and R5 are identical or different and represent a hydrogen atom, a substituted or unsubstituted alkyl group, a substituted or unsubstituted aryl group, a substituted or unsubstituted aralkyl group or a heterocyclic group, or R4 and R5 represent the atoms necessary for forming, with the nitrogen, a ring;

The Dye₁ and Dye₂ groups are as defined for DYE.

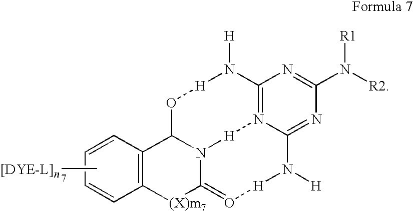

n₇ is at least 1;

m₇ is equal to 0 or 1;

For m₇=1, X represents O, NR3, or (CH₂)ₚ in which p is equal to 0, 1 or 2;

R1 and R2 are identical or different and represent a hydrogen atom, a substituted or unsubstituted alkyl group, a substituted or unsubstituted aryl group, a substituted or unsubstituted aralkyl group, a substituted or unsubstituted acyl group, a substituted or unsubstituted sulphonyl group or a heterocyclic group, or R1 and R2 represent the atoms necessary for forming a system with a nucleus;

R3 represents a hydrogen atom, a substituted or unsubstituted alkyl group, a substituted or unsubstituted aryl group or a substituted or unsubstituted aralkyl group;

The DYE group is as defined above and comprises a linker arm;

L represents a linker arm.

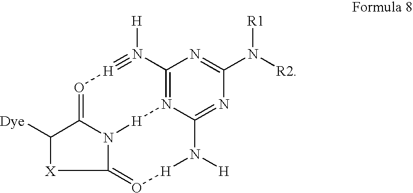

X represents O, NR3 or (CH₂)ₙ wherein n is at least 1;
R1 and R2 are identical or different and represent a hydrogen atom, a substituted or unsubstituted alkyl group, a substituted or unsubstituted aryl group, a substituted or unsubstituted aralkyl group, a substituted or unsubstituted acyl group, a substituted or unsubstituted sulphonyl group or a heterocyclic group, or R1 and R2 represent the atoms necessary for forming, with the nitrogen atom, a ring;
R3 represents a hydrogen atom, a substituted or unsubstituted alkyl group, a substituted or unsubstituted aryl group or a substituted or unsubstituted aralkyl group;
the DYE group is as defined above.

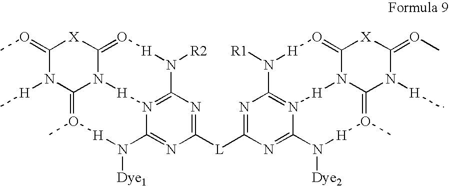

in which the Dye₁ and Dye₂ groups are as defined for “DYE”;
R1 and R2 are identical or different and represent a hydrogen atom, a substituted or unsubstituted alkyl group, a substituted or unsubstituted aryl group, a substituted or unsubstituted aralkyl group, a substituted or unsubstituted acyl group, a substituted or unsubstituted sulphonyl group, a substituted or unsubstituted cycloalkyl group or a heterocyclic group;
X represents NR3 or CR4R5; R3 represents a hydrogen atom, a substituted or unsubstituted alkyl group, a substituted or unsubstituted aryl group, a substituted or unsubstituted aralkyl group, a substituted or unsubstituted cycloalkyl group or a heterocyclic group;
R4 and R5 are identical or different and represent a hydrogen atom, a substituted or unsubstituted alkyl group, a substituted or unsubstituted aryl group, a substituted or unsubstituted aralkyl group or a heterocyclic group, or R4 and R5 represent the atoms necessary for forming, with the nitrogen atom, a ring;
L represents a linker arm as defined above.

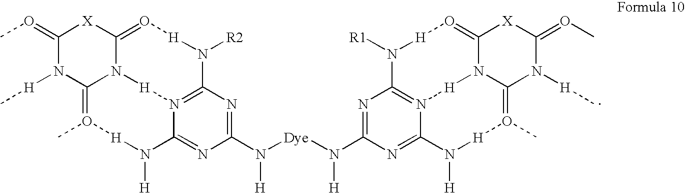

in which the “DYE” group is as defined above; R1 and R2 are identical or different and represent a hydrogen atom, a substituted or unsubstituted alkyl group, a substituted or unsubstituted aryl group, a substituted or unsubstituted aralkyl group, a substituted or unsubstituted acyl group, a substituted or unsubstituted sulphonyl group, a substituted or unsubstituted cycloalkyl group or a heterocyclic group;
X represents NR3 or CR4R5; R3 represents a hydrogen atom, a substituted or unsubstituted alkyl group, a substituted or unsubstituted aryl group, a substituted or unsubstituted aralkyl group, a substituted or unsubstituted cycloalkyl group or a heterocyclic group;
R4 and R5 are identical or different and represent a hydrogen atom, a substituted or unsubstituted alkyl group, a substituted or unsubstituted aryl group, a substituted or unsubstituted aralkyl group or a heterocyclic group, or R4 and R5 represent the atoms necessary for forming, with the nitrogen atom, a ring.

TABLE 1

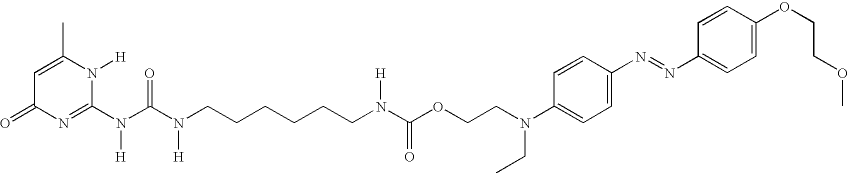

Dye 1

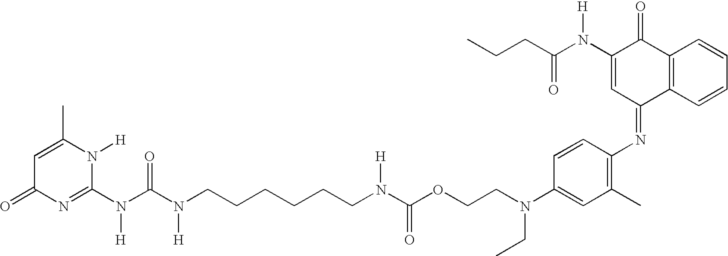

Dye 2

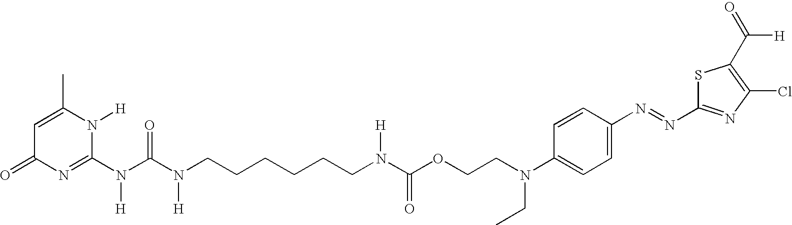

Dye 3

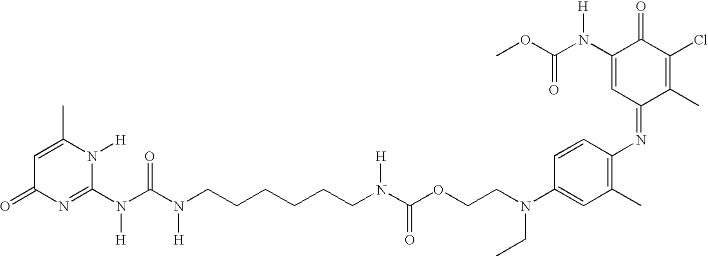

Dye 4

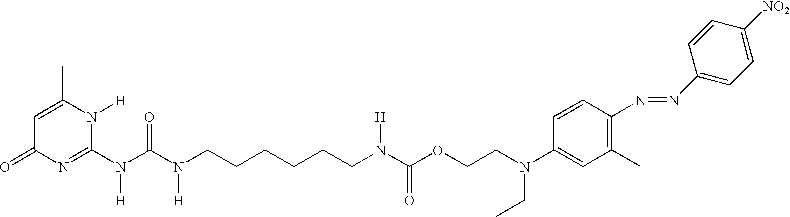

Dye 5

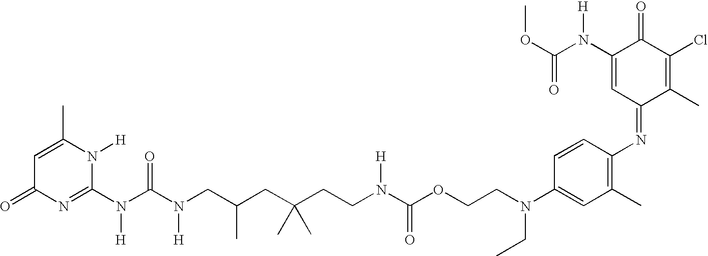

Dye 6

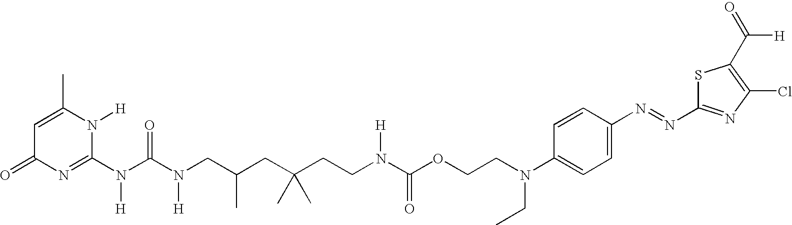

Dye 7

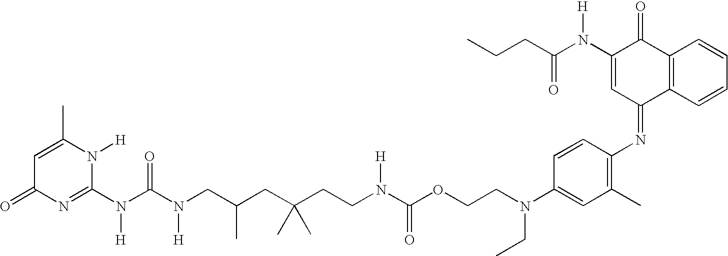

Dye 8

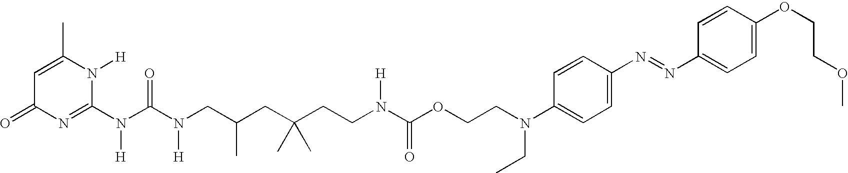

Dye 9

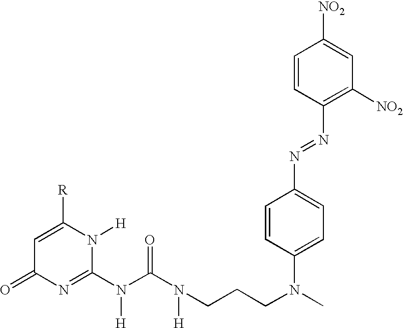

Dye 10

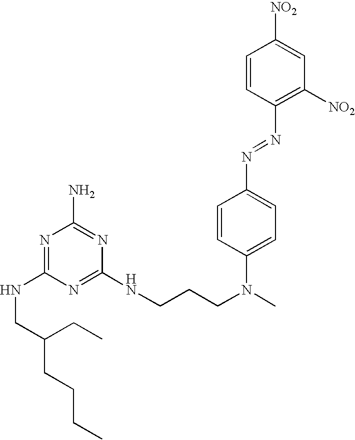

Dye 11

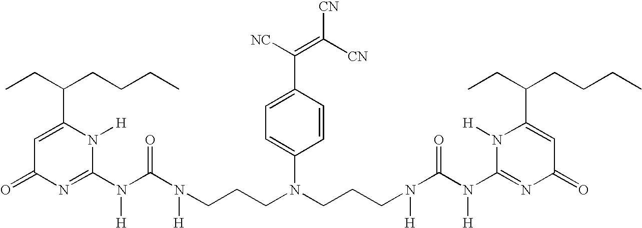

Dye 12

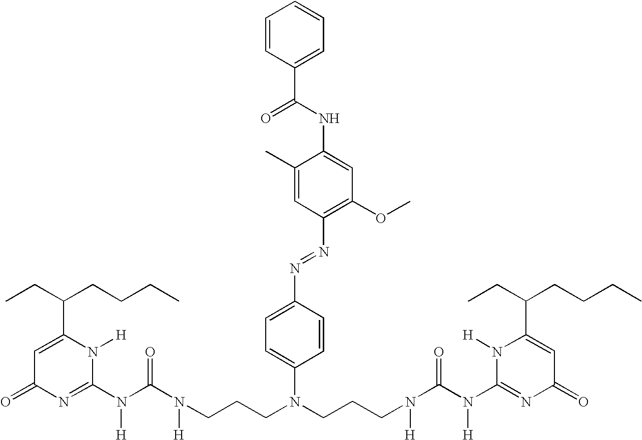

Dye 13

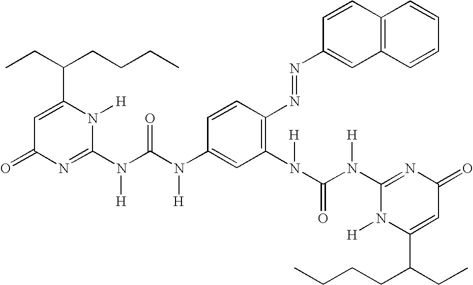

Dye 14

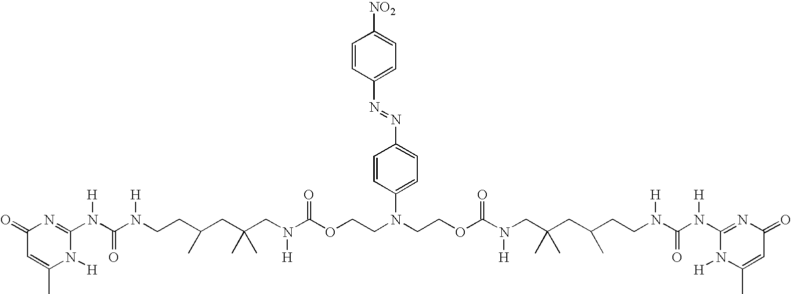

Dye 15

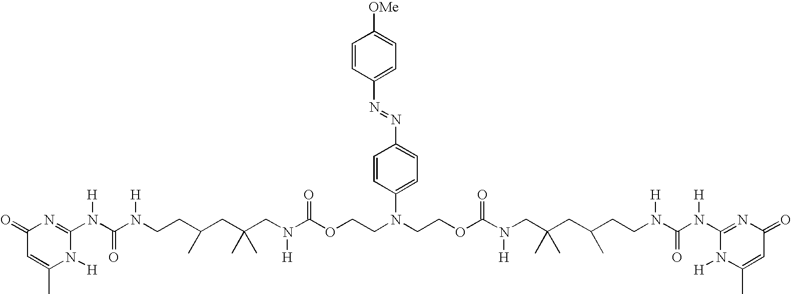

Dye 16

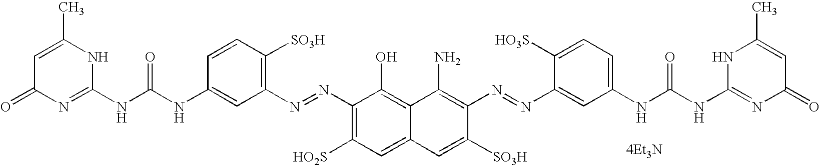

Dye 17

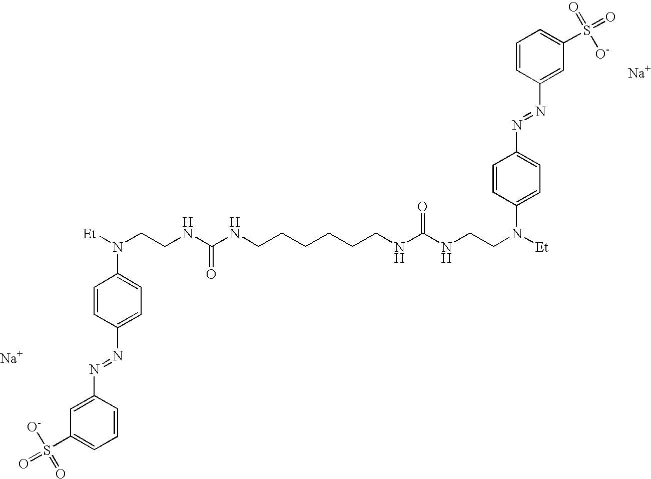

Dye 18

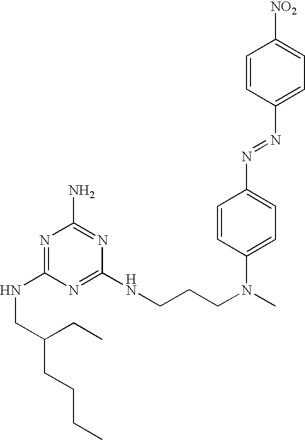

Dye 19

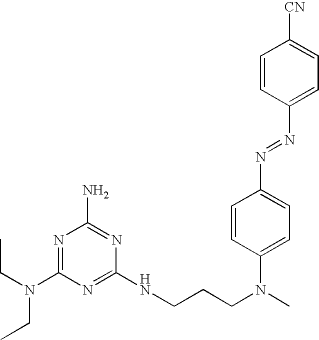

Dye 20

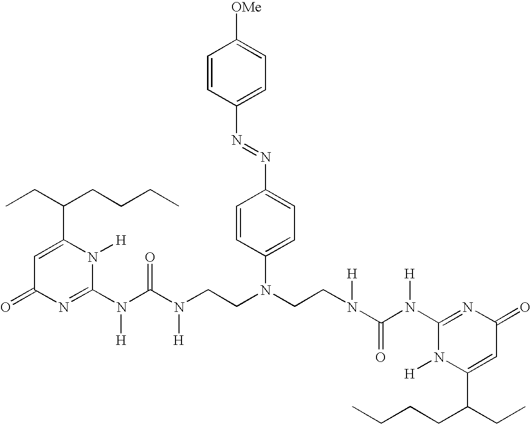

Dye 21

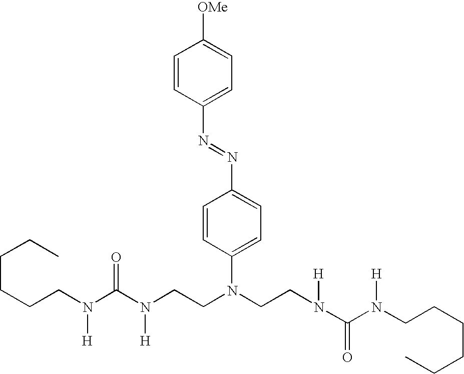

Dye 22

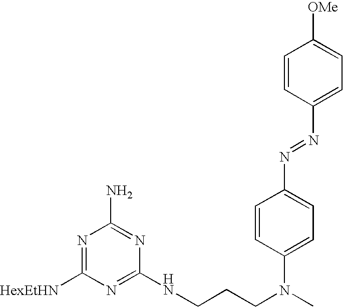

Dye 23

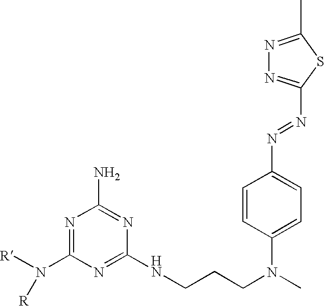

Dye 24 R, R′ = isobutyl
Dye 25 R = ethylhexyl, R′ = H

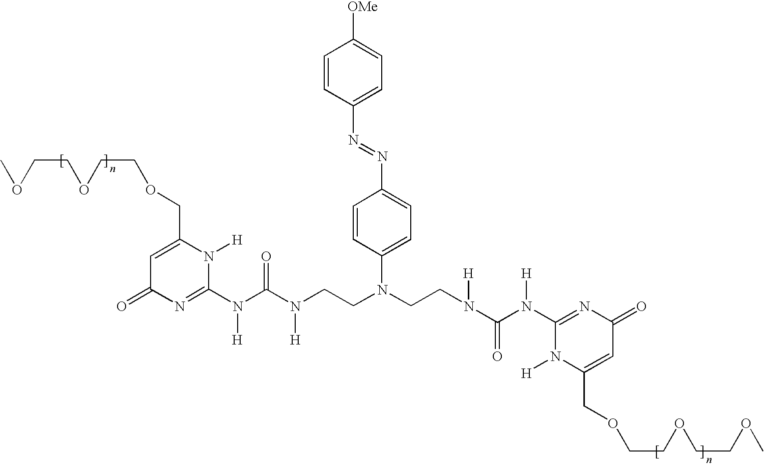

Dye 26 n = 2

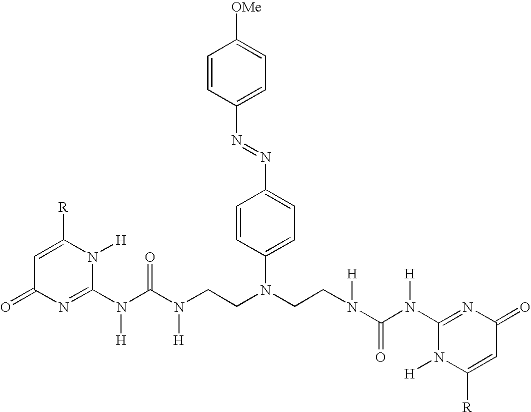

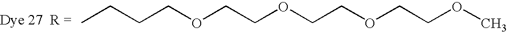

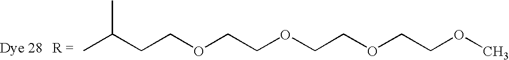

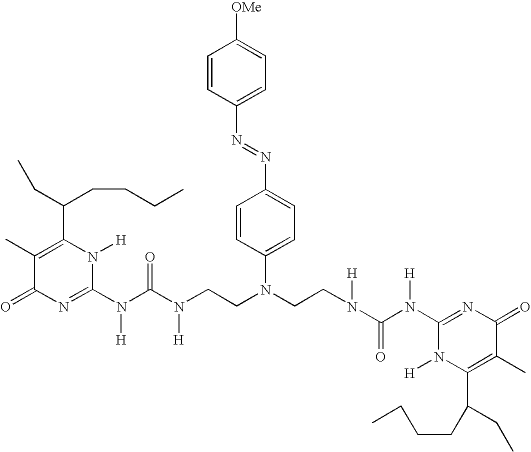

Dye 29

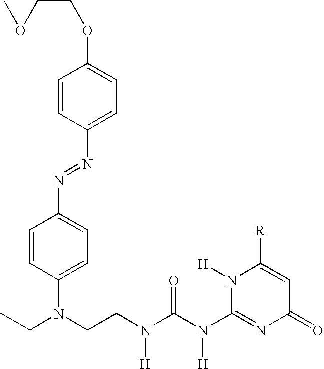

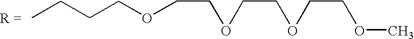

Dye 30

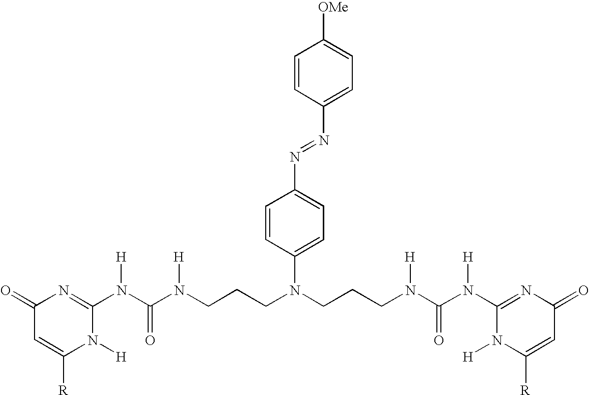

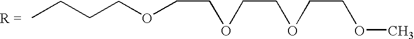

Dye 31

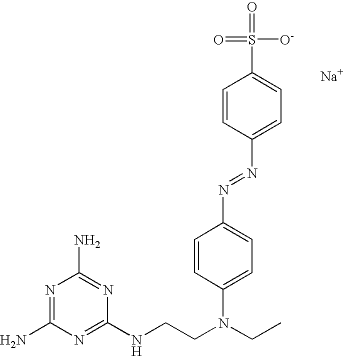

Dye 32

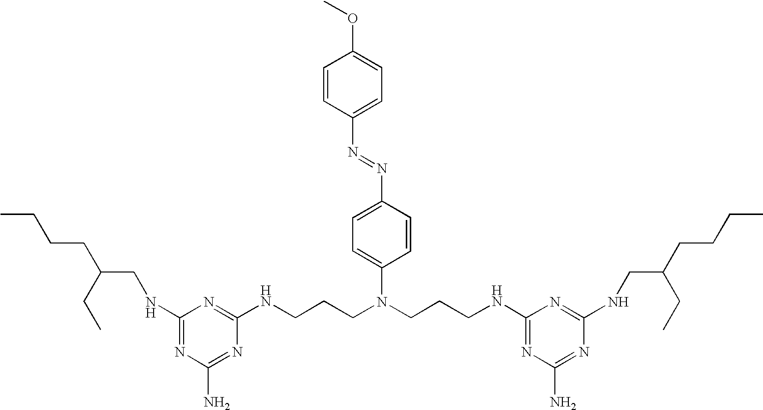

Dye 33

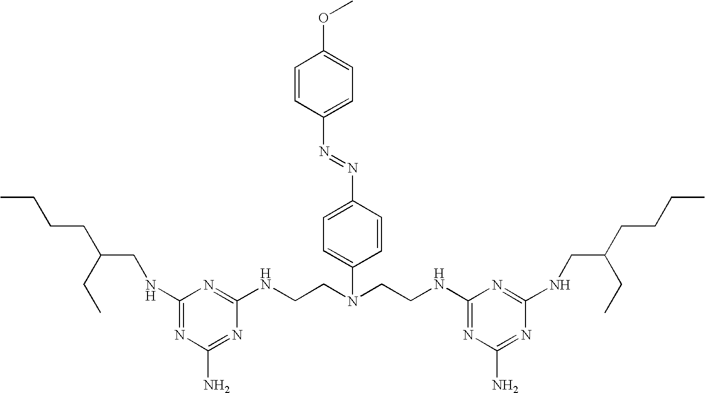

Dye 34

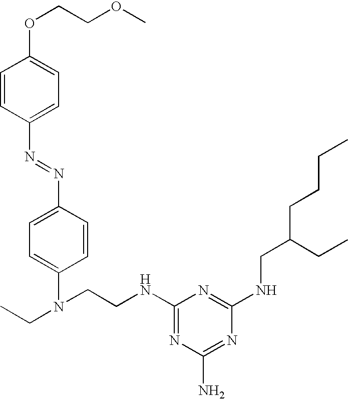

Dye 35

According to one variant, the colouring compositions contain at least one (DYE)ₙ(SAU)ₘ dye according to Formula (I) and at least one other dye, (DYE′)_{n′}(SAU′)_{m′}, chosen such that the (DYE)ₙ(SAU)ₘ dye(s) and the (DYE′)_{n′}(SAU′)_{m′} dye(s) are capable of assembling with one another.

According to another variant, the colouring compositions contain solely at least one (DYE)ₙ(SAU)ₘ dye according to Formula (I), it being possible for DYE to be identical or different.

In another embodiment of this invention, compositions are formulated which contain dyes according to Formula (I) and/or (I′) and compounds of formula (II) by which the (SAU) and/or (SAU′) and (SAU″) residues are chosen such that the dye(s) of formula (I) or (I′) and the compounds of formula (II) are capable of assembling with one another and of self-assembling.

The colouring compositions of the invention generally contain from 0.01% to 40%, preferably from 0.05% to 15%, and in particular from 0.5% to 5% by weight of the supra-molecular colouring system, relative to the total weight of the composition.

The appropriate medium for the colouring is generally constituted of water or comprises water and at least one organic solvent for solubilizing the compounds which would not be sufficiently soluble in the water. By way of an organic solvent, mention may, for example, be made of C₁-C₄ lower alkanols, such as ethanol and isopropanol; polyols and polyol ethers, such as 2-butoxyethanol, propylene glycol, propylene glycol monomethyl ether, diethylene glycol monoethyl ether and diethylene glycol monomethyl ether, and also aromatic alcohols such as benzyl alcohol or phenoxyethanol, and mixtures thereof.

The solvents are preferably present in proportions of preferably between approximately 1% and 40% by weight, relative to the total weight of the dyeing composition, and even more preferably between approximately 5% and 30% by weight.

The dyeing composition in accordance with the invention may also contain various adjuvants conventionally used in compositions for colouring the hair, such as anionic, cationic, nonionic, amphoteric or zwitterionic surfactants, or mixtures thereof, anionic, cationic, nonionic, amphoteric or zwitterionic polymers, or blends thereof, inorganic or organic thickeners, and in particular anionic, cationic, nonionic and amphoteric polymeric associative thickeners, antioxidants, penetrating agents, sequestering agents, fragrances, buffers, dispersants, conditioning agents, such as, for example, modified or non-modified, volatile or non-volatile silicones, film-forming agents, ceramides, preservatives, opacifiers, oils or waxes.

The above adjuvants are in general present in an amount, for each of them, of between 0.01% and 20% by weight, relative to the weight of the composition.

The pH of the dyeing composition in accordance with the invention is generally between approximately 3 and 12, and preferably between approximately 5 and 9. It may be adjusted to the desired value by means of acidifying or basifying agents normally used in the colouring of keratinous material, in particular the hair, or alternatively using conventional buffer systems.

Among the acidifying agents, mention may be made, by way of example, of inorganic or organic acids, such as hydrochloric acid, orthophosphoric acid or sulphuric acid, carboxylic acids, such as acetic acid, tartaric acid, citric acid or lactic acid, and sulphonic acids.

Among the basifying agents, mention may be made, by way of example, of aqueous ammonia, alkali carbonates, alkanolamines, such as mono-, di- and triethanolamines, and also derivatives thereof, sodium hydroxide or potassium hydroxide and the compounds of formula (III) below:

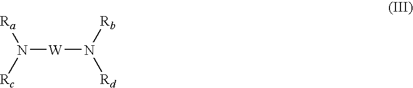

in which W is a propylene residue optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

According to one particular embodiment of colouring medium, mention may be made of the following medium:

Alkyl (C₈/C₁₀ 50/50) polyglucoside as a
buffered 60% aqueous solution

Benzyl alcohol
Polyethylene glycol 400 comprising 8
ethylene oxide units
Aqueous ammonia at 20% and/or
monoethanolamine and/or urea
Water

For dyes which are more hydrophobic, mention may in particular be made of tripropylene glycol monomethyl ether, propylene glycol n-propyl ether, monoalkyl or dialkyl ether derivatives of tri- or dipropylene glycol, propylene glycol methyl ether acetate, and propylene glycol phenyl ether.

The dyeing composition according to the invention may be in various forms, such as in the form of liquids, creams or gels, or in any other form suitable for colouring keratinous material, and in particular human hair.

For colouring keratin fibres, the colouring method is such that the composition according to the present invention as defined above is applied to the fibres for a period of time sufficient to obtain the desired effect.

The material to which the composition is applied may be dry or wet.

The colouring of the keratinous material is obtained when the supra-molecular systems form by formation of hydrogen bonds.

The formation of the supra-molecular colouring system can be obtained spontaneously, by evaporation, for example, of the water contained in the colouring medium. This formation of the supra-molecular colouring system can be promoted by the presence of specific compounds in the colouring medium, for example volatile compounds, or by rinsing, by washing or by heating.

The keratinous material thus coloured can then be rinsed, washed and dried.

Such an application can be carried out in the presence of an oxidizing agent, it being possible for this oxidizing agent to be applied simultaneously with or prior to the application of the composition of the invention in order to obtain prior or simultaneous lightening of the keratin fibre. According to one particular embodiment, the oxidizing agent is present in the composition of the invention.

The oxidizing agents conventionally used for the oxidation dyeing of keratin fibres are, for example, hydrogen peroxide, urea peroxide, alkali metal bromates, persalts such as perborates and persulphates, peracids and oxidase enzymes, among which mention may be made of peroxidases, 2-electron oxidoreductases such as uricases, and 4-electron oxygenases such as laccases. Hydrogen peroxide is particularly preferred.

A subject of the invention is also a kit which contains at least a first composition containing one or more compounds of formula (I), (I′) or (II) or (II′) and a second composition containing an oxidizing agent. According to one variant, the kit also comprises a composition containing the alkaline agent.

1-23. (canceled)

24. A cosmetic coloring composition comprising in a cosmetically acceptable medium, a coloring system comprising at least one dye having formula (I):
(DYE)ₙ(SAU)ₘ (I)
wherein,
for each occurrence, (DYE) is covalently bonded to (SAU) and comprises at least one chromophore having a maximum absorption from 300 nm to 1200 nm;
for each occurrence, (SAU) comprises a radical independently chosen from aminopyrimidones, ureidopyrimidons, trimellitic acid, acylaminopyridines, ureidopyridines, carbamoylpyridines, acylaminotriazines, ureidotriazines, ureidoaminotriazines, acylaminotriazines, acylaminotriazoles, urazoylbenzoic acid, phthalhydrazides, 2-aminopyrimidine-4,6(1H,5H)-diones, and 4,6-diaminopyrimidin-2(1H)-one, each of which may be substituted at the 5-position with at least one group chosen from:
linear, branched or cyclic, C₁-C₂₀ optionally substituted saturated or unsaturated alkylene radicals, optionally substituted with one or more heteroatoms;
optionally substituted cycloalkylene radical;
optionally substituted arylene radical;
saturated or unsaturated heterocyclic radical; and
amino, O, SO, SO₂, and C(O);
n and m are each integers independently chosen from values greater than or equal to 1; and
wherein said (SAU) radical is capable of forming at least three hydrogen bonds and is capable of self-assembly.

25. A cosmetic coloring composition according to claim 24, wherein, for each occurrence, (DYE) comprises at least one chromophore having a maximum absorption from 380 nm to 850 nm.

26. A cosmetic coloring composition according to claim 24, wherein, for each occurrence, (DYE) comprises at least one chromophore chosen from acridines, acridones, anthranthrones, anthrapyrimidines, anthraquinones, azines, azos, azomethines, benzanthrones, benzimidazoles, benzimidazolones, benzindoles, benzoxazoles, benzopyranes, benzothiazoles, benzoquinones, bisazines, bis isoindolines, carboxanilides, coumarines, cyanines, diazines, diketopyrrolopyrroles, dioxazines, diphenylamines, diphenylmethanes, dithiazines, flavonoides, fluorindines, formazans, hydrazones, hydroxyketones, indamines, indanthrones, indigoides, pseudoiondigoides, indophenols, indoanilines, isoindolines, isoindolinones, isoviolanthrones, lactones, methines, naphthalimides, naphthanilides, naphtholactames, naphthoquinones, nitro, oxadiazoles, oxazines, perilones, périnones, perylenes, phenazines, phenothiazines, phthalocyanine, polyenes, carotenoides, porphyrines, pyranthrones, pyrazolanthrones, pyrazolones, pyrimidinoanthrones, pyronines, quinacridones, quinolines, quinophthalones, squaranes, stilbènes, thiazines, thioindigo, thiopyronines, triarylmethanes, and xanthenes.

27. A cosmetic coloring composition according to claim 24, wherein the (SAU) radical is capable of forming 3 or 4 hydrogen bonds.

28. A cosmetic coloring composition according to claim 24, wherein the at least one dye of Formula (I) is chosen from dyes of Formula 1: 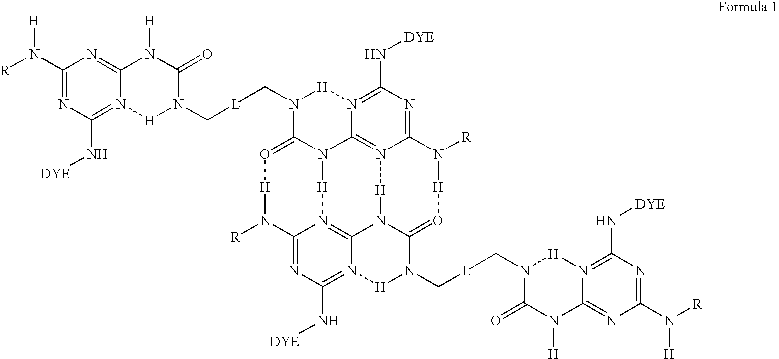
wherein,
for each occurrence, R is independently chosen from hydrogen, alkyl, substituted alkyl, aryl, substituted aryl, aralkyl, substituted aralkyl, acyl, substituted acyl, sulphonyl, substituted sulphonyl, phosphoryl, substituted phosphoryl, and a heterocyclic group; and
for each occurrence, L is independently chosen from
linear, branched or cyclic C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O).

29. A cosmetic coloring composition according to claim 24, wherein the at least one dye of Formula (I) is chosen from dyes of Formula 2: 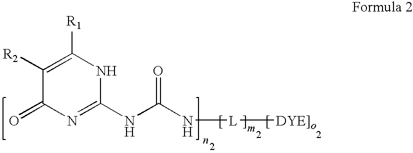
wherein,
for each occurrence, R₁ and R₂ are independently chosen from hydrogen, halogen, alkoxy, substituted alkoxy, thioalkoxy, substituted thioalkoxy, sulphoxy, substituted sulphoxy, sulphone, substituted sulphone, amino, substituted amino, nitrile, alkyl, unsaturated alkyl, alkyl, substituted unsaturated alkyl, acyl, substituted acyl, sulphonyl, substituted sulphonyl, aryl, substituted aryl, aralkyl, substituted aralkyl, heterocyclic group, and a DYE group;
for each occurrence, n₂ and o₂ are independently chosen from integers greater than or equal to 1;
for each occurrence, m₂ independently is an integer greater than or equal to 0; and
for each occurrence, L is independently chosen from
linear, branched or cyclic, C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O).

30. A cosmetic coloring composition according to claim 24, wherein the at least one dye of Formula (I) is chosen from dyes of Formula 3: 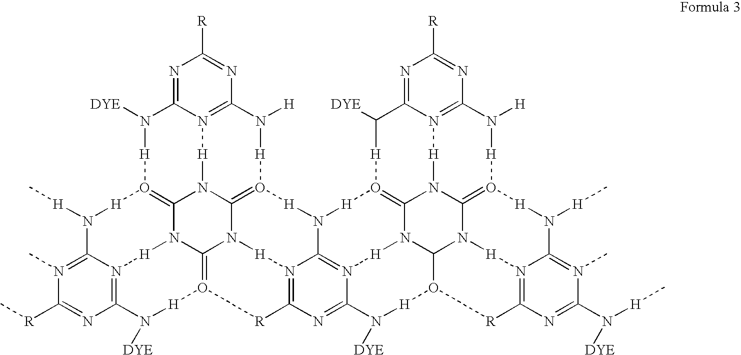
wherein,
for each occurrence, R is independently chosen from hydrogen, alkyl, substituted alkyl, aryl, substituted aryl, aralkyl, substituted aralkyl, a heterocyclic group, a DYE group, OR₁, and NR₂R₃ wherein:
for each occurrence, R₁ is independently chosen from hydrogen, alkyl, substituted alkyl, acyl, substituted acyl, sulphonyl, substituted sulphonyl, aryl, substituted aryl, aralkyl, substituted aralkyl, a heterocyclic group, and a DYE group; and
for each occurrence, R₂ and R₃ are independently chosen from hydrogen, alkyl, substituted alkyl, aryl, substituted aryl, aralkyl, substituted aralkyl, a heterocyclic group, acyl, substituted acyl, sulphonyl, substituted sulphonyl, and a DYE group wherein R₂ and R₃ may form with the nitrogen atom to which they are attached a ring.

31. A cosmetic coloring composition according to claim 24, wherein the at least one dye of Formula (I) is chosen from dyes of Formula 5: 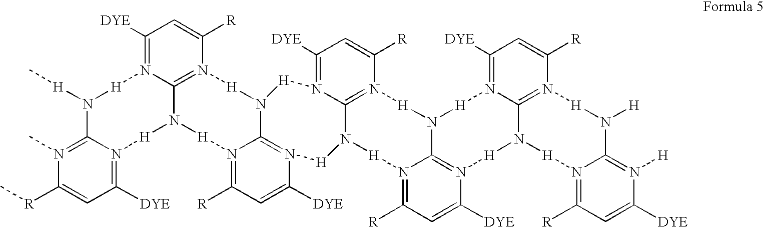
wherein,
for each occurrence, R is independently chosen from hydrogen, alkyl, substituted alkyl, aryl, substituted aryl, aralkyl, substituted aralkyl, a heterocyclic group, a DYE group, OR₁ and NR₂R₃;
for each occurrence, R₁ is independently chosen from hydrogen, alkyl, substituted alkyl, aryl, substituted aryl, aralkyl, substituted aralkyl, and a heterocyclic group; and
for each occurrence, R₂ and R₃ are independently chosen from hydrogen, alkyl, substituted alkyl, aryl, substituted aryl, aralkyl, substituted aralkyl, and a heterocyclic group, wherein R₂ and R₃ may form with the nitrogen atom to which they are attached a ring.

32. A cosmetic coloring composition according to claim 24, wherein the at least one dye of Formula (I) is chosen from dyes of Formula 6: 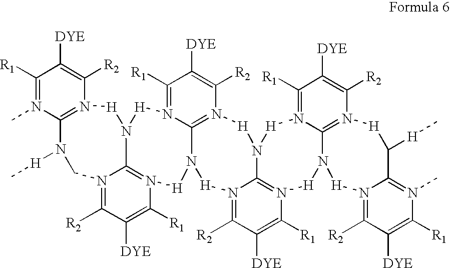
wherein,
for each occurrence, R₁ and R₂ are independently chosen from hydrogen, alkyl, substituted alkyl, aryl, substituted aryl, aralkyl, substituted aralkyl, a heterocyclic group, OR₃ and NR₄R₅; and
for each occurrence, R₄ and R₅ are independently chosen from hydrogen, alkyl, substituted alkyl, aryl, substituted aryl, aralkyl, substituted aralkyl, and a heterocyclic group wherein R₄ and R₅ may form with the nitrogen atom to which they are attached a ring.

33. A cosmetic coloring composition according to claim 24, wherein the at least one dye of Formula (I) is chosen from dyes of Formula 9: 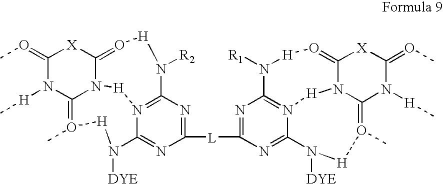
wherein,
for each occurrence, R₁ and R₂ are independently chosen from hydrogen, alkyl, substituted alkyl, aryl, substituted aryl, aralkyl, substituted aralkyl, sulphonyl, substituted sulphonyl, cycloalkyl, substituted cycloalkyl, and a heterocyclic group;
for each occurrence, X is independently chosen from NR₃ or CR₄R₅;
for each occurrence, R₃ is independently chosen from hydrogen, alkyl, substituted alkyl, aryl, substituted aryl, aralkyl, substituted aralkyl, cycloalkyl, substituted cycloalkyl, and a heterocyclic group;
for each occurrence, R₄ and R₅ are independently chosen from hydrogen, alkyl, substituted alkyl, aryl, substituted aryl, aralkyl, substituted aralkyl, and a heterocyclic group wherein R₄ and R₅ may form with the nitrogen atom to which they are attached a ring; and
for each occurrence, L is chosen from
linear, branched or cyclic C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O).

34. A cosmetic coloring composition according to claim 24, wherein the at least one dye of Formula (I) is chosen from dyes of Formula 10: 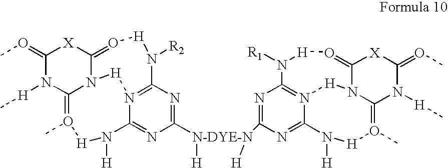
wherein,
for each occurrence, R₁ and R₂ are independently chosen from hydrogen, alkyl, substituted alkyl, aryl, substituted aryl, aralkyl, substituted aralkyl, acyl, substituted acyl, sulphonyl, substituted sulphonyl, cycloalkyl, substituted cycloalkyl, and a heterocyclic group;
for each occurrence, X is independently chosen from NR₃ or CR₄R₅;
for each occurrence, R₃ is independently chosen from hydrogen, alkyl, substituted alkyl, aryl, substituted aryl, aralkyl, substituted aralkyl, cycloalkyl, substituted cycloalkyl, and a heterocyclic group; and
for each occurrence, R₄ and R₅ are independently selected from hydrogen, alkyl, substituted alkyl, aryl, substituted aryl, aralkyl, substituted aralkyl, and a heterocyclic group wherein R₄ and R₅ may form with the nitrogen atom to which they are attached a ring.

35. A cosmetic coloring composition according to claim 24, comprising from 0.01% to 40% by weight of the coloring system, relative to the total weight of the cosmetic coloring composition.

36. A cosmetic coloring composition according to claim 35, comprising from 0.05% to 15% by weight of the coloring system, relative to the total weight of the cosmetic coloring composition.

37. A cosmetic coloring composition according to claim 24, wherein said cosmetically acceptable medium is chosen from water, organic solvent, and volatile compounds.

38. A cosmetic coloring composition comprising in a cosmetically acceptable medium, a coloring system comprising at least one dye having the Formula 4: 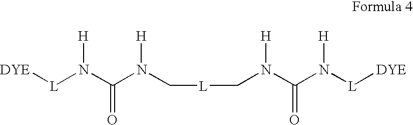
wherein,
for each occurrence, DYE is at least one chromophore having a maximum absorption from 300 nm to 1200 nm;
for each occurrence, L is independently chosen from
linear, branched or cyclic C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O).

39. A cosmetic coloring composition according to claim 38, comprising from 0.01% to 40% by weight of the coloring system, relative to the total weight of the cosmetic coloring composition.

40. A cosmetic coloring composition according to claim 39, comprising from 0.05% to 15% by weight of the coloring system, relative to the total weight of the cosmetic coloring composition.

41. A cosmetic coloring composition according to claim 38, wherein said cosmetically acceptable medium is chosen from water, organic solvent, and volatile compounds.

42. A cosmetic coloring composition, comprising in a cosmetically acceptable medium, a supra-molecular coloring system comprising at least one dye having the
Formula 7: 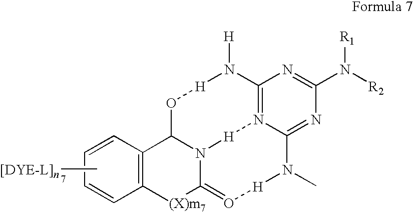
wherein,
for each occurrence, DYE is independently chosen from at least one chromophore having a maximum absorption from 300 nm to 1200 nm;
for each occurrence, L is independently chosen from
linear, branched or cyclic C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O);
for each occurrence, R₁ and R₂ are independently chosen from hydrogen, alkyl, substituted alkyl, aryl, substituted aryl, aralkyl, substituted aralkyl, sulphonyl, substituted sulphonyl, and a heterocyclic group;
for each occurrence, R₃ is independently chosen from hydrogen, alkyl, substituted alkyl, aryl, substituted aryl, and aralkyl, substituted aralkyl;
for each occurrence, n₇ is independently an integer of at least one;
for each occurrence, m₇ is independently 0 or 1; and
if m₇=1, then X is chosen from O, NR₃, or (CH₂)ₚ wherein p is equal to 1, 2, or 3.

43. A cosmetic coloring composition according to claim 42, comprising from 0.01% to 40% by weight of the coloring system, relative to the total weight of the cosmetic coloring composition.

44. A cosmetic coloring composition according to claim 43, comprising from 0.05% to 15% by weight of the coloring system, relative to the total weight of the cosmetic coloring composition.

45. A cosmetic coloring composition according to claim 42, wherein said cosmetically acceptable medium is chosen from water, organic solvent, and volatile compounds.

46. A cosmetic coloring composition, comprising in a cosmetically acceptable medium, a coloring system comprising at least one dye having Formula 8: 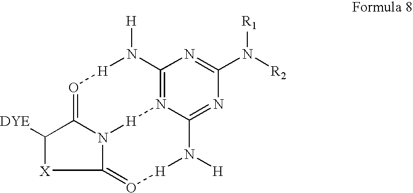
wherein,
DYE is chosen from at least one chromophore having a maximum absorption from 300 nm to 1200 nm;
X is chosen from O, NR₃, and (CH₂)ₙ;
n is at least one;
R₁ and R₂ are chosen from hydrogen, alkyl, substituted alkyl, aryl, substituted aryl, aralkyl, substituted aralkyl, acyl, substituted acyl, sulphonyl, substituted sulphonyl, and a heterocyclic group; and
R₃ is chosen from hydrogen, alkyl, substituted alkyl, aryl, substituted aryl, aralkyl, and substituted aralkyl.

47. A cosmetic coloring composition according to claim 46, comprising from 0.01% to 40% by weight of the coloring system, relative to the total weight of the cosmetic coloring composition.

48. A cosmetic coloring composition according to claim 47, comprising from 0.05% to 15% by weight of the coloring system, relative to the total weight of the cosmetic coloring composition.

49. A cosmetic coloring composition according to claim 46, wherein said cosmetically acceptable medium is chosen from water, organic solvent, and volatile compounds.

50. A method for coloring keratinous materials comprising,
applying to said keratinous materials a coloring system comprising at least one dye having formula (I):
(DYE)ₙ(SAU)ₘ (I)
wherein,
for each occurrence, (DYE) is covalently bonded to (SAU) and comprises at least one chromophore having a maximum absorption from 300 nm to 1200 nm;
for each occurrence, (SAU) comprises a radical chosen from aminopyrimidones, ureidopyrimidons, trimellitic acid, acylaminopyridines, ureidopyridines, carbamoylpyridines, acylaminotriazines, ureidotriazines, ureidoaminotriazines, acylaminotriazines, acylaminotriazoles, urazoylbenzoic acid, phthalhydrazides, 2-aminopyrimidine-4,6(1H,5H)-diones, and 4,6-diaminopyrimidin-2(1H)-one, each of which may be substituted at the 5-position with at least one group chosen from:
linear, branched or cyclic C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O);
n and m are each integers independently chosen from values greater than or equal to 1, wherein said (SAU) radical is capable of forming at least three hydrogen bonds and is capable of self-assembly; and
applying said coloring system for a period of time to obtain coloration.

51. A method according to claim 27, wherein said keratinous materials are hair.

52. A kit comprising a coloring system comprising at least one dye having formula (I):
(DYE)ₙ(SAU)ₘ (I)
wherein,
for each occurrence, (DYE) is covalently bonded to (SAU) and comprises at least one chromophore having a maximum absorption from 300 nm to 1200 nm;
for each occurrence, (SAU) comprises a radical chosen from aminopyrimidones, ureidopyrimidons, trimellitic acid, acylaminopyridines, ureidopyridines, carbamoylpyridines, acylaminotriazines, ureidotriazines, ureidoaminotriazines, acylaminotriazines, acylaminotriazoles, urazoylbenzoic acid, phthalhydrazides, 2-aminopyrimidine-4,6(1H,5H)-diones, and 4,6-diaminopyrimidin-2(1H)-one, each of which may be substituted at the 5-position with at least one group chosen from:
linear, branched or cyclic C₁-C₂₀ optionally substituted saturated or unsaturated alkylene radicals, wherein the alkylene radicals are further optionally substituted with one or more heteroatoms;
optionally substituted cycloalkylene radicals;
optionally substituted arylene radicals;
saturated or unsaturated heterocyclic radicals;
amino;
O;
SO;
SO₂; and
C(O);
n and m are each integers independently chosen from values greater than or equal to 1, wherein said (SAU) radical is capable of forming at least three hydrogen bonds and is capable of self-assembly; and
a second composition comprising an oxidizing agent.

53. A kit according to claim 29, further comprising an alkaline agent.

54. A cosmetic coloring composition, comprising in a cosmetically acceptable medium, a coloring system comprising at least one dye chosen from 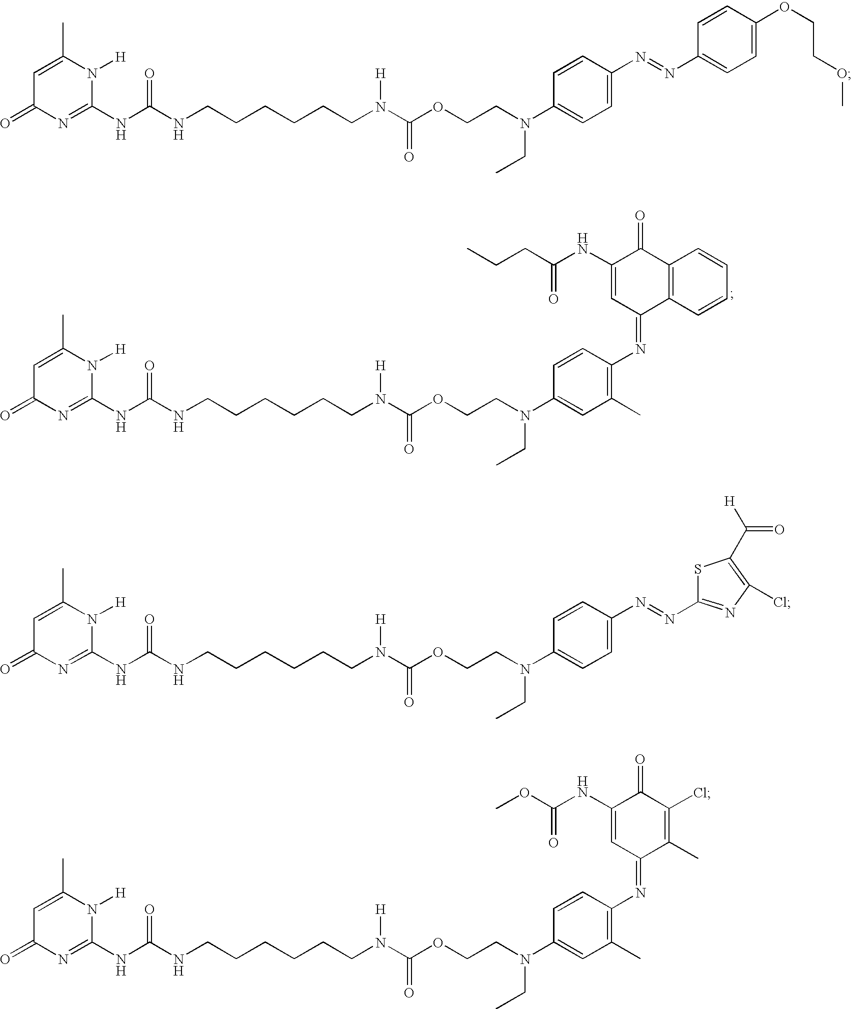 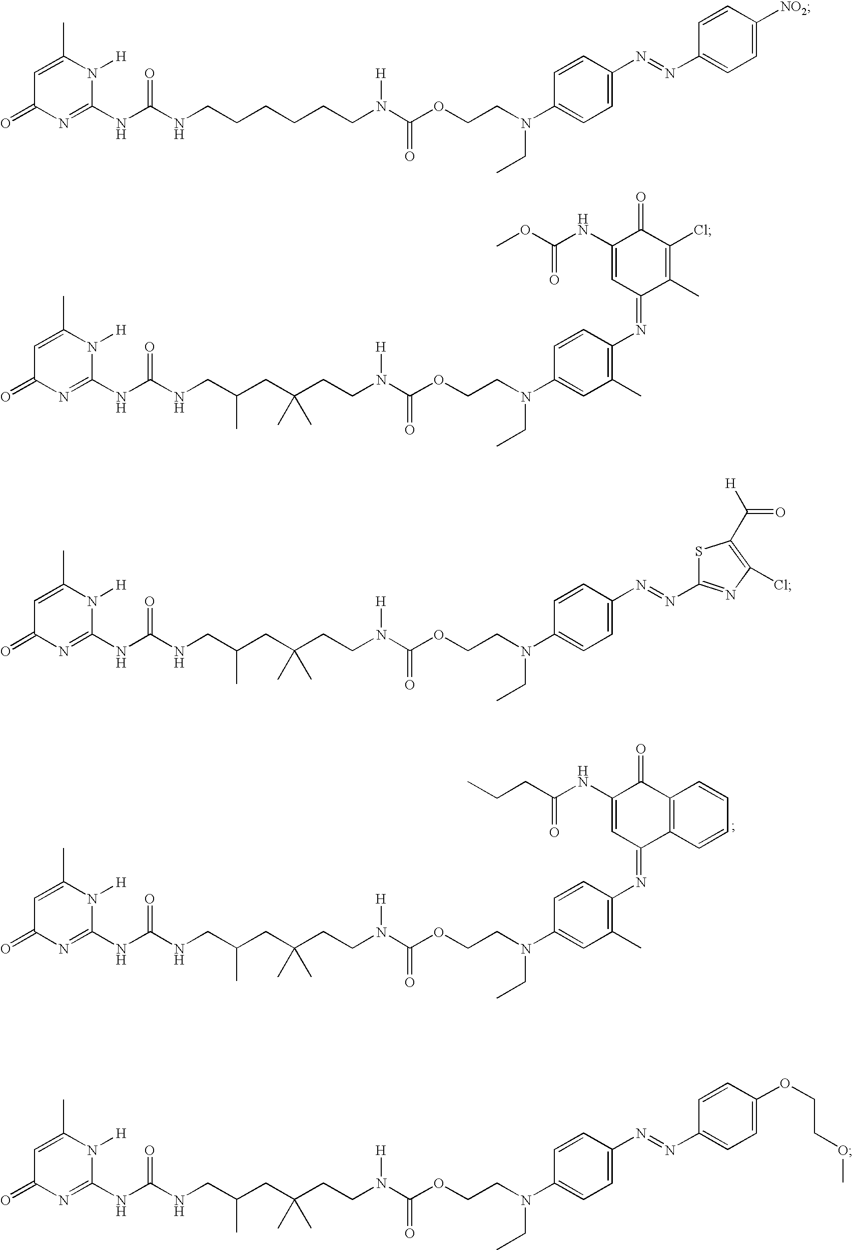 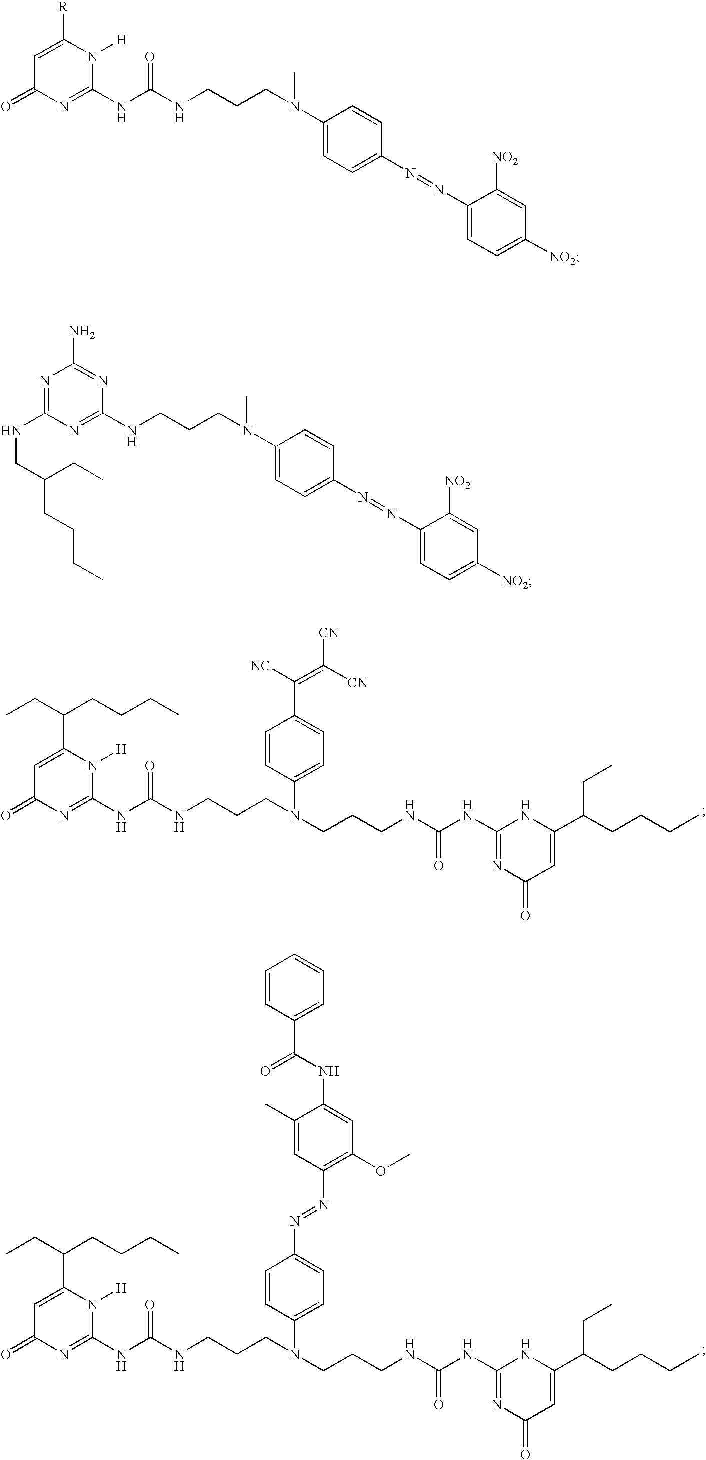 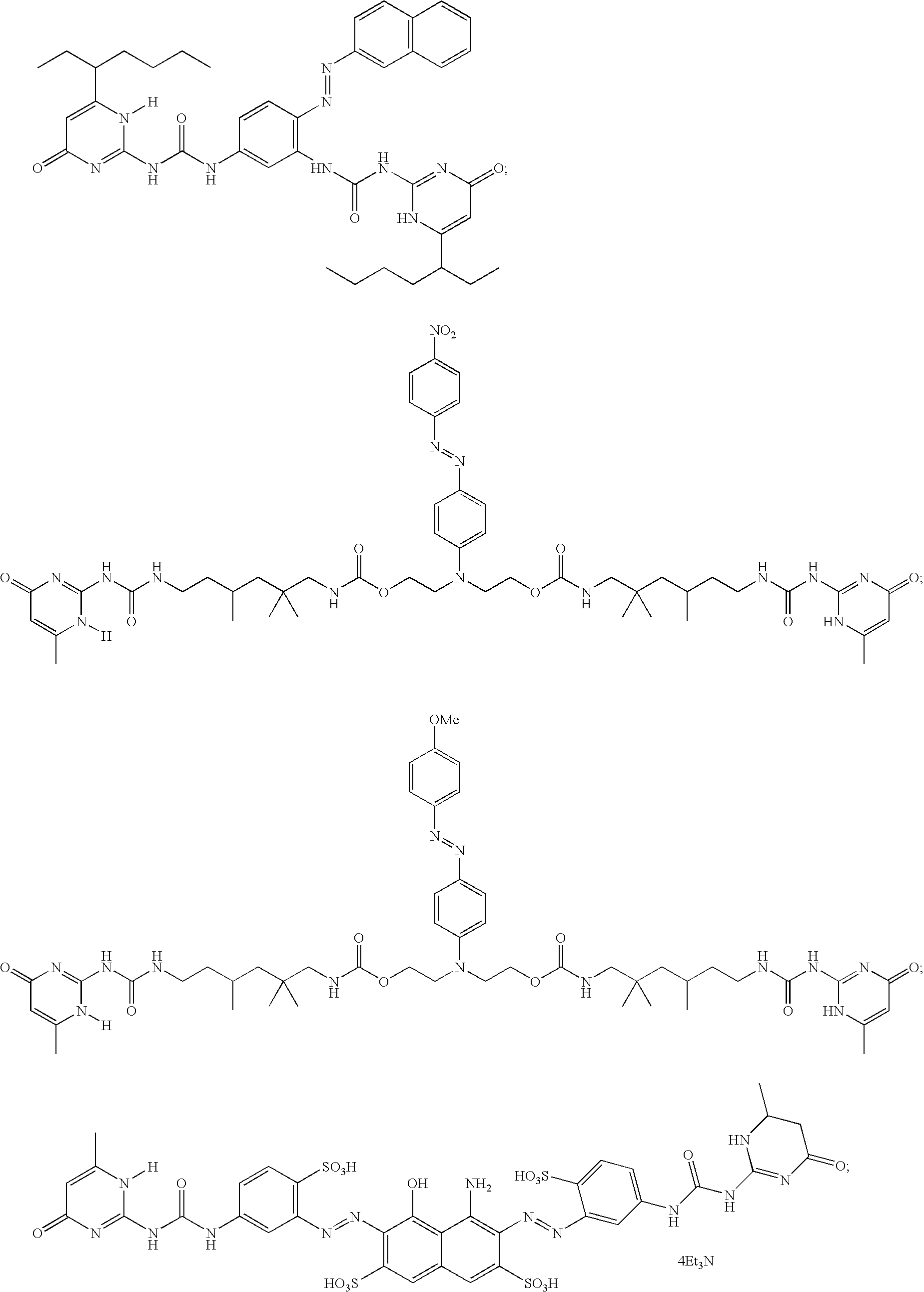 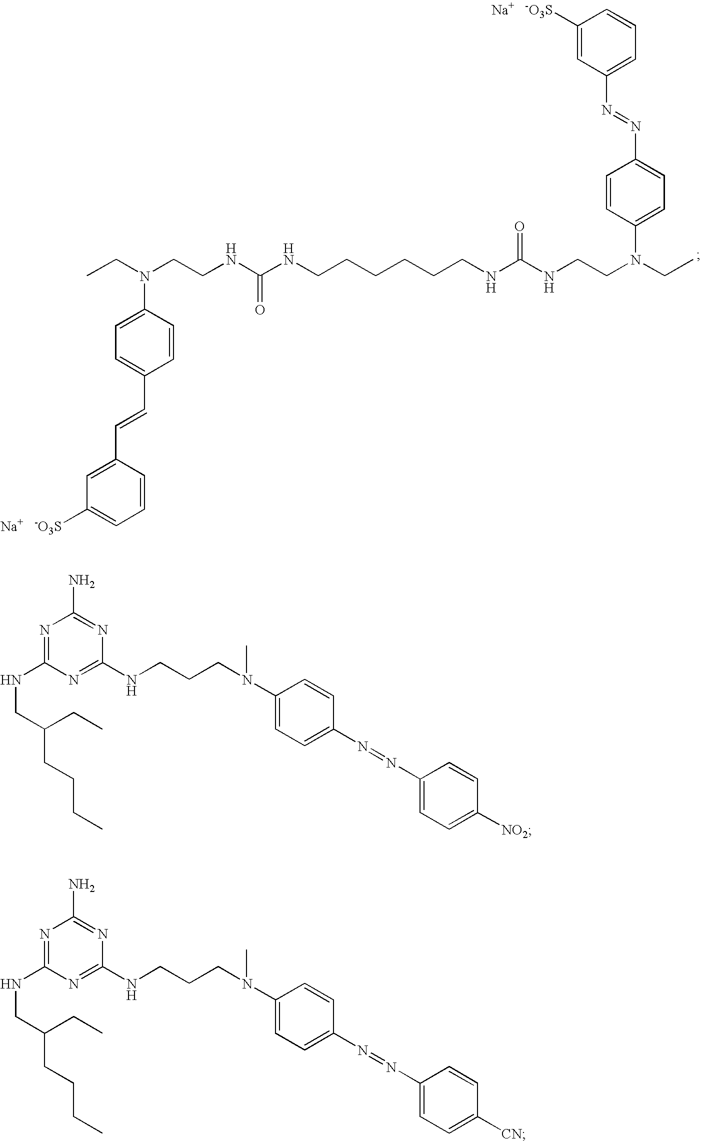 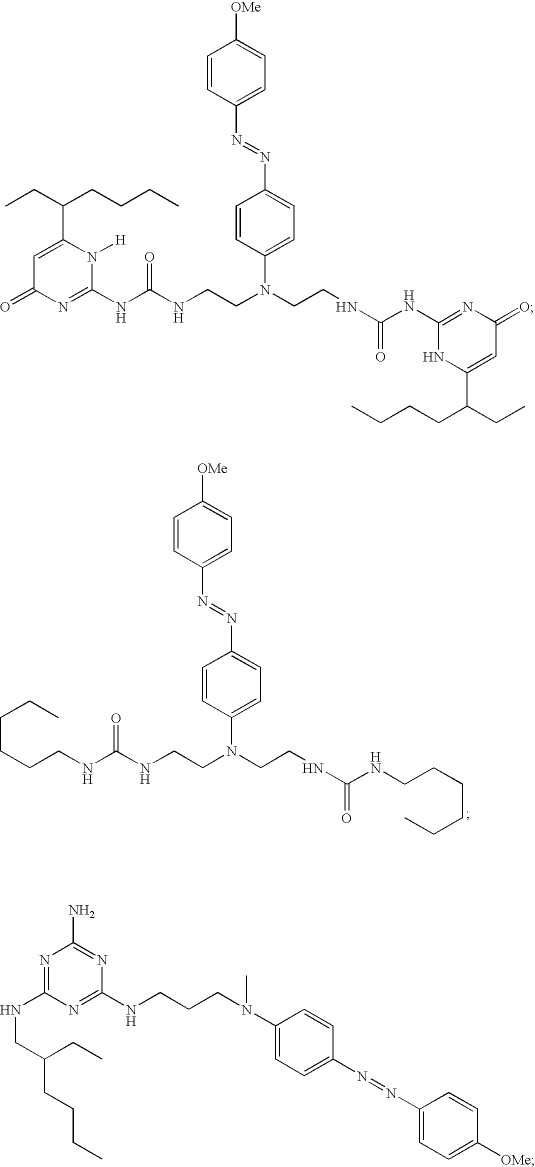 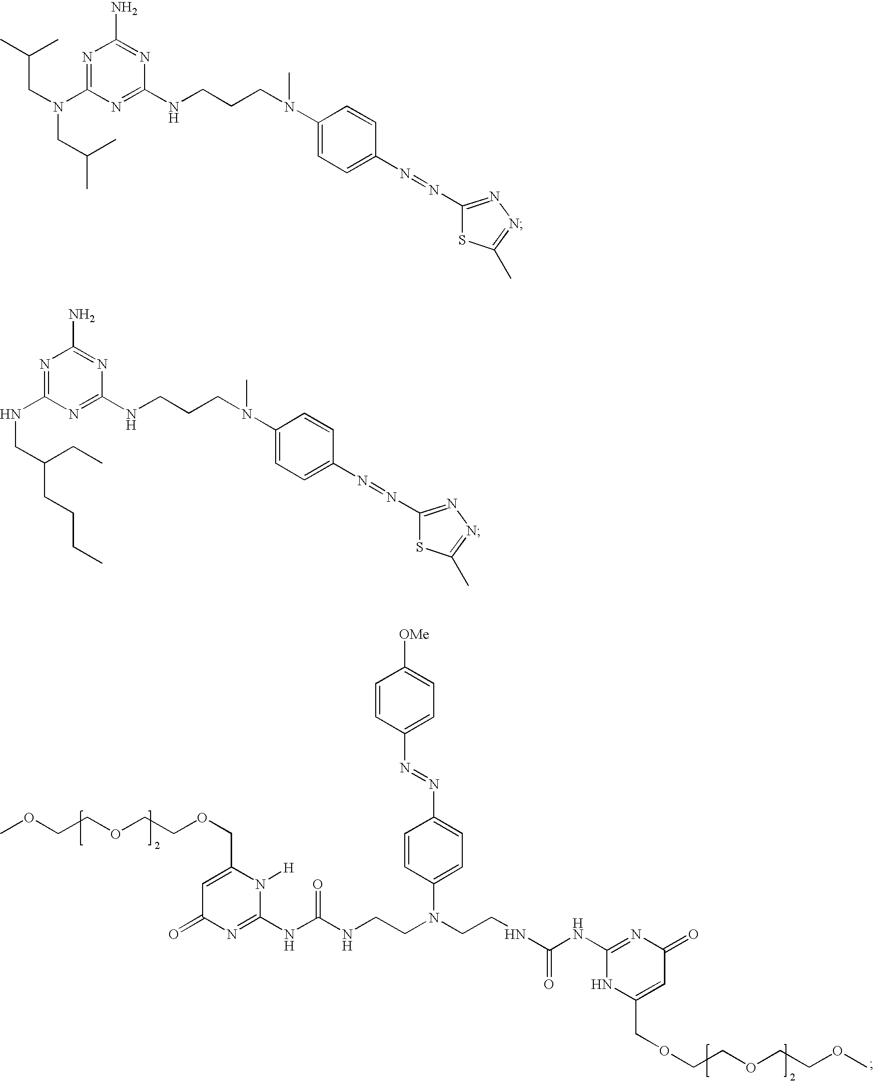 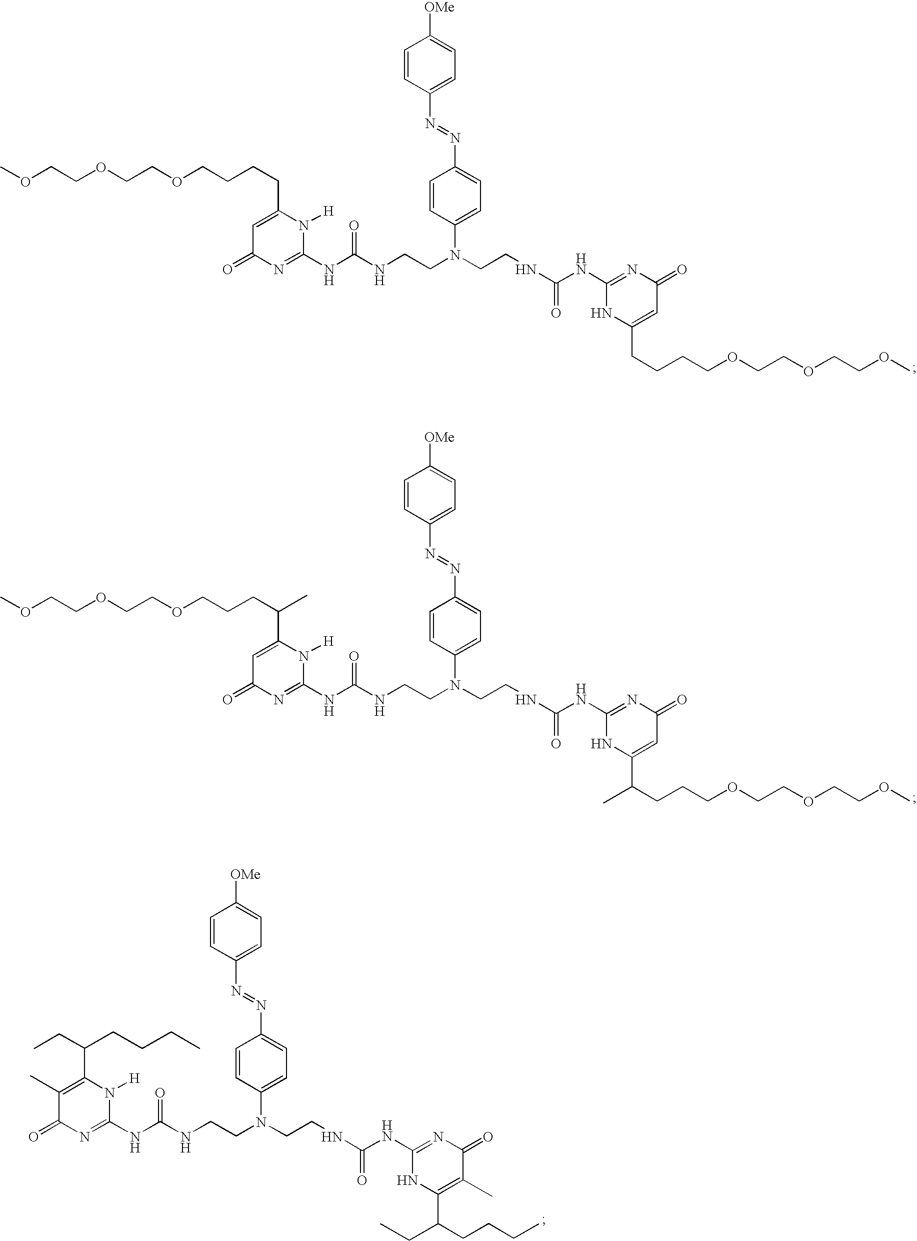 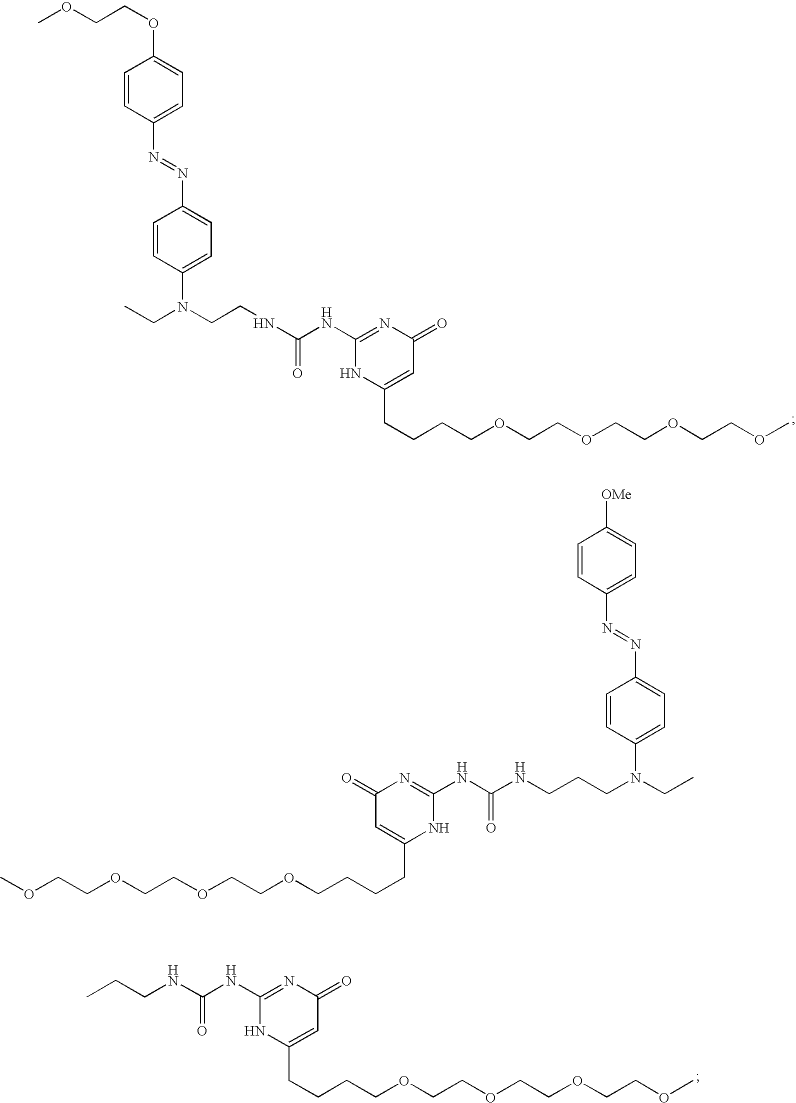 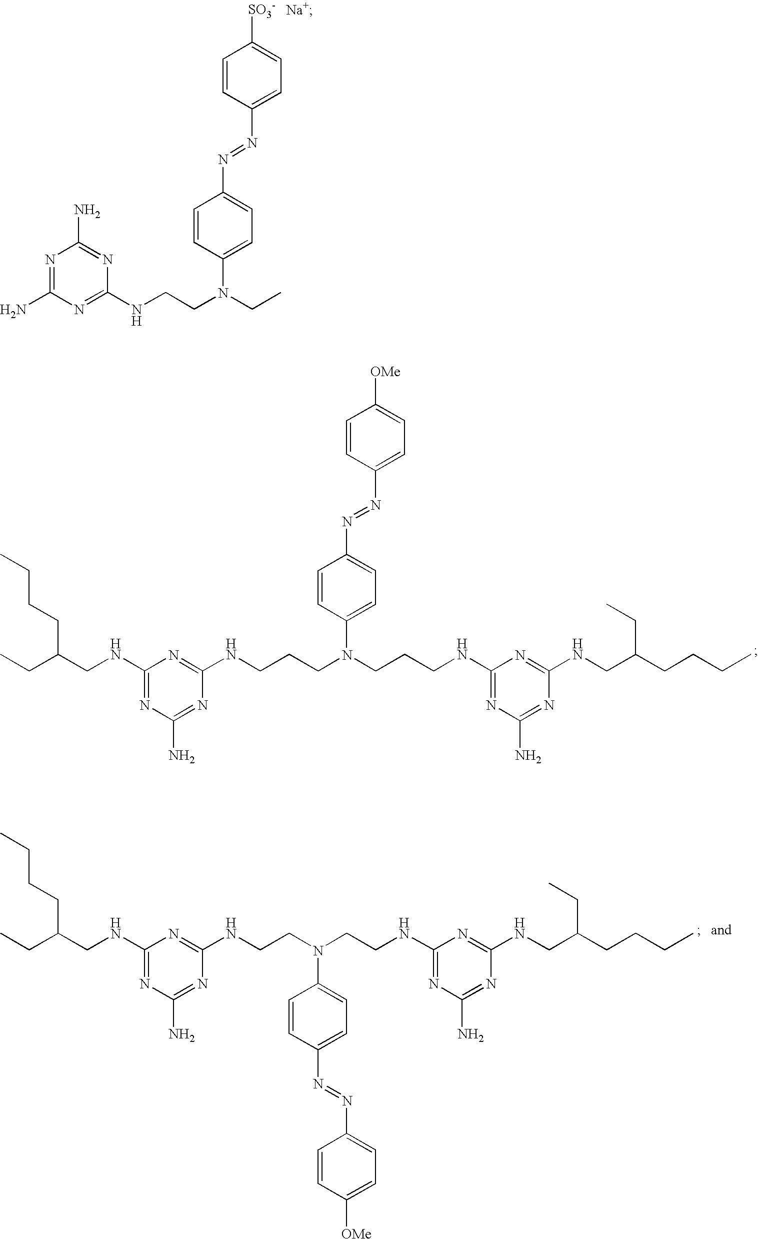 [chemical structure]
